(19) United States
(12) Patent Application Publication
(10) Pub. No.: US 2025/0170236 A1
(43) Pub. Date: May 29, 2025

(54) A NOVEL CATIONIC ADJUVANT COMPOSITION

(71) Applicant: STATENS SERUM INSTITUT, Copenhagen S (DK)

(72) Inventors: Joshua WOODWORTH, Copenhagen S (DK); Gabriel PEDERSEN, Charlottenlund (DK); Dennis CHRISTENSEN, Frederiksberg (DK); Rasmus MORTENSEN, Hvidovre (DK)

(73) Assignee: STATENS SERUM INSTITUT, Copenhagen S (DK)

(21) Appl. No.: 18/840,175

(22) PCT Filed: Feb. 21, 2023
(86) PCT No.: PCT/EP2023/054284

§ 371 Date: Aug. 21, 2024

(30) Foreign Application Priority Data
Feb. 21, 2022 (EP) 22157663.0

(51) Int. Cl. A61K 39/39; A61K 39/00; A61K 39/04; A61K 39/118; A61K 39/145; A61P 31/06; A61P 31/16; C12N 7/00

(52) U.S. Cl. CPC A61K 39/39; A61K 39/04; A61K 39/118; A61K 39/145; A61P 31/06; A61P 31/16; C12N 7/00; A61K 2039/545; A61K 2039/55555; A61K 2039/55561; A61K 2039/575; C12N 2760/16134

(57) ABSTRACT

The present invention relates to an adjuvant composition comprising dimethyldioctadecyl ammonium salt (DDA), monomycoloyl glycerol (MMG), and the CpG ODN 2006 oligodeoxynucleotide having SEQ ID NO:1 or a sequence having 90% identity to SEQ ID NO:1. Another aspect of the present invention is a vaccine comprising said adjuvant composition and at least one antigen, and the use of said vaccine in prevention or treatment of an infectious disease.

### TECHNICAL FIELD OF THE INVENTION

The present invention relates to a novel adjuvant composition as well as a vaccine comprising the novel adjuvant. In particular, the present invention relates to an adjuvant composition comprising a CpG oligonucleotide, in particular CpG ODN 2006 (CpG2006), and the liposomal components dimethyldioctadecyl ammonium salt (DDA) and monomycolyl glycerol (MMG), which act synergistically to increase the immunogenic response of vaccines.

### BACKGROUND OF THE INVENTION

The majority of novel generation vaccines are based on highly pure proteins or peptides derived from the pathogen. However, due to the inherently low immunogenicity of proteins and peptides, a major focus has been directed towards designing adjuvants that serve to enhance the immune response of the vaccine. Although a number of new adjuvant systems have been identified during the past 30 years, the need for new adjuvant systems is still recognized, which is evident in the paucity of choices available for clinical use.

An adjuvant (from latin adjuvare, to help) can be defined as any substance that when administered in the vaccine serves to direct, accelerate, prolong and/or enhance the specific immune response. Depending on the nature of the adjuvant it can promote a cell-mediated immune response, a humoral immune response or a mixture of the two. When used as a vaccine adjuvant, an antigenic component is added to the adjuvant. Since the enhancement of the immune response mediated by adjuvants is non-specific, it is well understood in the field that the same adjuvant can be used with different antigens to promote responses against different targets e.g. with an antigen from M. tuberculosis to promote immunity against M. tuberculosis or an antigen from C. trachomatis to promote immunity against chlamydia.

Many of the remaining disease targets for which there are presently no effective vaccines rely on varying levels of cell-mediated immune (CMI) responses with or without an associated humoral response. Tuberculosis (TB), HIV, malaria and chlamydia all belong to this category of global health problems that are crucially dependent on CMI responses and—for the latter two—also humoral responses for protection. But, also many of the existing vaccines may benefit from an improved adjuvant technology stimulating both arms of the immune system. This is illustrated by influenza and coronavirus, where antibodies neutralize the infectivity of the virus and T-cells reduce severity and provide cross-strain protection thereby serving to reduce disease and enhance recovery from infection.

Recently, it has also become evident that antibodies not only neutralize, e.g. virus, but can also regulate immune responses by interacting with Fc receptors on the surface of innate immune cells. In particular, the IgG2 subclasses (IgG2b and IgG2a/c) in mice have been associated with the most potent antibody responses against viruses and intracellular bacteria. Hence, vaccine-induced IgG2 has distinct properties to IgG1 and is important, e.g. as a part of the antiviral immune response. Although it is not possible to identify a human analogue, human IgG3 shares many characteristics with mouse IgG2. The higher activity of IgG2 has attracted a lot of interest in chlamydia, where this isotype is found responsible for antibody enhancement of Th1 activation and the subsequent protection. Over the last 20 years, there has been a breakthrough in our understanding of how the various antibody isotypes interact with either activating or inhibitory Fc receptors and thereby mediate the differential activity observed in vivo. Thus, IgG1 antibodies selectively bind to inhibitory FcγRIIB receptors expressed on dendritic cells, whereas IgG2 antibodies preferentially engage with the activating FCγRIV receptor crucial for the higher in vivo activity observed as e.g. enhanced phagocytosis, antibody dependent cellular cytotoxicity (ADCC) and release of inflammatory mediators. There is therefore a growing interest for both the quality and the quantity of the vaccine-induced antibody response, which has crucial importance for the development of the cellular immune response and thereby the protective or therapeutic properties of a vaccine. An adjuvant that induces a high amount of antibodies engaging with activating receptors will therefore be very valuable in this context.

Presently, only a few adjuvants are widely accepted for human use, e.g. aluminium-based adjuvants (AlOH-salts) and squalene emulsions e.g. MF59 and AS03. Both of these adjuvant types are inducers of a humoral immune response but provide relatively poor cell-mediated immunity (CMI). As the generation of a robust CMI response is considered essential, e.g. for a protective immune response against many pathogens, novel adjuvants have been developed and some are now licensed and used for their ability to increase CMI responses. These include AS01 (liposomes comprising the immunostimulatory components QS21 and Monophosphoryl Lipid A (MPL; TLR-4 agonist)) in Shingrix and CpG ODN 1018 in Heplisav B. Both adjuvants are used in high doses causing side effects in many individuals.

CpG ODN's stimulate immune responses by binding to toll-like receptor 9. This receptor is expressed in various forms in different immune cells. There are many different CpG ODN's in clinical development in vaccines, typically given in doses of 0.5-3 mg/dose but in some cases at even higher doses up to 20 mg. These CpGs show a clear dose dependency with respect to immune responses, however, dose-related side effects also occur. A few studies have shown that combining the CpG with a delivery system can reduce the needed CpG dose, and thus potentially also the dose-related side effects including systemic inflammatory responses.

Dimethyldioctadecylammonium bromide, -chloride, -phosphate, -acetate or other organic or inorganic salts (DDA) is a lipophilic quaternary ammonium compound, which forms cationic liposomes in aqueous solutions at temperatures above ˜40° C. DDA has been used extensively as an adjuvant, e.g. the administration of Arquad 2HT, which comprises DDA, in humans was promising and did not induce apparent side effects. The combination of DDA and immunomodulators as adjuvants have been described e.g. DDA and TDB, DDA and MMG or DDA and MPL which all showed a very clear synergy enhancing the immune response compared to the responses obtained with either DDA alone or the immunomodulator alone. DDA-based formulations are therefore promising adjuvants candidates for inclusion in vaccines. The combination of cationic liposomes (e.g. DDA) and a non-ionic surfactant has been used in an oil emulsion delivering drugs to cells. Furthermore, cationic amphiphiles and non-ionic surfactants have been used separately to form mixtures of cationic liposomes and neutral liposomes to target tumour cells with greater efficiency compared to cationic liposomes alone.

From immunogenicity studies in mice, it is known that the combination of DDA and MMG (CAF®04) as an adjuvant induces a Th1/17 type of immune response characterized by substantial production of IFNγ and IL-17 and at the same time levels of IgG1 and IgG2 comparable to what is observed using DDA alone. Recent studies furthermore showed that the combination of CAF®04 with a specific CpG (ODN 1826) can increase the CMI responses significantly but does not increase the antibody responses. Whilst the increased CMI response is intriguing, an optimized adjuvant, which can boost both CMI responses and antibody responses is desired. Hence, an adjuvant composition, obtained by combining CAF®04 with an additional immunostimulator, which is able to increase both IgG (and especially IgG2b or a/c) and Th1/17 cell responses over CAF®04, would be advantageous. Such an adjuvant combination would be particularly attractive if it did not induce systemic side-effects associated with immune reactions to free (unbound) immunostimulator. The adjuvant would be attractive in prophylactic subunit-based vaccines against a number of infectious diseases including TB, Chlamydia, Streptoccoci, influenza A and coronaviruses.

### SUMMARY OF THE INVENTION

Thus, an object of the present invention is to improve the vaccine-induced antibody response, which has crucial importance for the development of the cellular immune response and thereby the protective or therapeutic properties of a vaccine.

In particular, an object of the present invention is to provide an adjuvant composition, which is able to increase both IgG (and especially IgG2b or a/c) and Th1/17 cell responses and which at the same time do not induce systemic side-effects associated with immune reactions to free (unbound) immunostimulator.

Thus, an aspect of the present invention relates to an adjuvant composition comprising dimethyldioctadecyl ammonium salt (DDA), monomycoloyl glycerol (MMG), and the CpG ODN 2006 oligodeoxynucleotide having the sequence SEQ ID NO: 1 or a sequence having 90% identity to SEQ ID NO: 1.

Another aspect of the present invention relates to a vaccine comprising or consisting of the adjuvant composition of the present invention and at least one antigen.

Yet another aspect of the present invention relates to the vaccine according to the present invention for use in prevention or treatment of infectious disease.

### BRIEF DESCRIPTION OF THE FIGURES

FIG. 1 shows that incorporating 10 μg CpG1826 into a murine dose of DDA/MMG liposomes corresponding to 2500/500/100 μg/ml DDA/MMG/CpG1826 does not increase the antibody response over DDA/MMG alone, using the M. tuberculosis H56 antigen.

FIG. 2 illustrates that the incorporation of 10 μg CpG2006 into a murine dose of DDA/MMG liposomes corresponding to 2500/500/100 μg/ml DDA/MMG/CpG2006 substantially increases the antibody response over DDA/MMG alone both at 15 days post immunization (FIG. 2A; Day 15) and 1 day after the 2nd immunization (FIG. 2B; Day 22) as well as over CpG2006 alone (FIG. 2C), using the H56 antigen.

FIG. 3 shows that a murine dose of DDA/MMG combined with 20 μg CpG2006 corresponding to 2500/500/200 μg/ml DDA/MMG/CpG2006, increases the IgG2c response, but not IgG1, over murine doses of DDA/MMG alone and DDA/MMG combined with 20 μg CpG1826, using the H107e antigen.

FIG. 4 shows that the incorporation of 10 μg CpG2006 into a murine dose of DDA/MMG liposomes corresponding to 2500/500/100 μg/ml DDA/MMG/CpG2006 substantially increases the T cell response over equal doses of DDA/MMG and CpG2006 alone, using the H56 antigen. This is similar to DDA/MMG/CpG1826.

FIG. 5 illustrates that the incorporation of 50 μg CpG2006 into a murine dose of DDA/MMG liposomes corresponding to 2500/500/500 μg/ml DDA/MMG/CpG2006 increases the antibody response compared to 50 μg CpG1826 in DDA/MMG liposomes at two time points, using the H56 antigen.

FIG. 6 shows that incorporation of CpG2006 into murine dose of DDA/MMG liposomes increases the IgG2c responses in doses ranging from 2-50 μg corresponding to 2500/500/20 μg/ml to 2500/500/500 μg/ml DDA/MMG/CpG2006, using the H56 antigen. The IgG1 responses are unaffected by the incorporation of CpG2006 into the DDA/MMG liposomes.

FIG. 7 shows that encapsulating CpG2006 in DDA/MMG liposomes reduces CpG-induced systemic inflammation, using the H56 antigen.

FIG. 8 illustrates that DDA/MMG liposomes are superior to DOTAP-liposomes for inducing antibody responses when combined with CpG2006, using the H56 antigen.

FIG. 9 shows that DDA/MMG/CpG2006 liposomes are more potent antibody inducers than the known adjuvant liposomes, DDA/MMG/poly (IC) (CAF®09b), with the M. tuberculosis H107 antigen in a non-human primate model.

FIG. 10 shows that DDA/MMG liposomes with 20 μg CpG2006 increase Th17 responses (FIG. 10A) and induce superior protection against M. tuberculosis infection with the H107 antigen compared to DDA/MMG with 20 μg CpG1826 (FIG. 10B).

FIG. 11 shows that DDA/MMG/CpG2006 liposomes induce long-lived antibody responses with the CTH522 antigen (FIG. 11A) and confer increased protection against a chlamydia infection compared to the known adjuvant liposome, DDA/MMG/poly (IC) (CAF®09b) (FIG. 11B).

FIG. 12 shows that a murine dose of DDA/MMG combined with 20 mg CpG2006 corresponding to 2500/500/200 μg/ml DDA/MMG/CpG2006, increases total IgG and IgG2c responses over murine doses of DDA/MMG combined with 20 mg CpG1826, using influenza Hemagglutinin antigen.

FIG. 13 shows that a murine dose of DDA/MMG combined with 20 mg CpG2006 corresponding to 2500/500/200 μg/ml DDA/MMG/CpG2006, increases Th17 responses over murine doses of DDA/MMG combined with 20 mg CpG1826, using influenza Hemagglutinin antigen.

### DETAILED DESCRIPTION OF THE INVENTION

### Definitions

Prior to discussing the present invention in further details, the following terms and conventions will first be defined:

### Liposome

The term “liposome” or “liposomal composition” is a broad definition for vesicles composed of lipid bilayers enclosing aqueous compartments. The membrane-forming lipids are amphiphilic and accordingly contain a polar and an apolar region. The polar region typically consists of a phosphate group, an acidic group and/or tertiary or quaternary ammonium salts and can either have a net negative (anionic), neutral or positive (cationic) surface charge at physiological pH, depending on the composition of the lipid head groups. The pH is preferably adjusted to physiological pH such as by dispersion adjusted to pH 5.0-8.0 in Tris or histidine buffer, most preferably adjusted to pH 6.5-7.5. The apolar region typically consists of one or more fatty acid chains with at least 8 carbons and/or cholesterol. The lipids constituting the vesicular bilayer membranes are organized such that the apolar hydrocarbon “tails” are oriented toward the centre of the bilayer while the polar “heads” orient towards the in- and outside aqueous phase, respectively.

Thus, “liposome” or “liposomal” is defined as closed vesicle structures made up of one or more lipid bilayers surrounding an aqueous core. Each lipid bilayer is composed of two lipid monolayers, each of which has a hydrophobic “tail” region and a hydrophilic polar “head” region. In the lipid bilayer, the hydrophobic “tails” of the lipid monolayers orient toward the inside of the bilayer, while the hydrophilic “heads” orient toward the outside of the bilayer. Liposomes can have a variety of physicochemical properties such as size, lipid composition, surface charge, fluidity and number of bilayer membranes. According to the number of lipid bilayers, liposomes can be categorized as unilamellar vesicles (UV) or small unilamellar vesicles (SUV) comprising a single lipid bilayer or multilamellar vesicles (MLV) comprising two or more concentric bilayers each separated from the next by a layer of water. Water soluble compounds are entrapped within the aqueous phases/core of the liposomes opposed to lipophilic compounds, which are trapped in the core/center of the lipid bilayer membranes.

The term “cationic lipid” or “cationic liposome” is intended to include any amphiphilic lipid, including natural as well as synthetic lipids and lipid analogs, having hydrophobic and polar head group moieties, a net positive charge at physiologically acceptable pH, and which can form bilayer vesicles or micelles in water.

### Antigen

In the present context, the term “antigen” refers to a molecule, such as an immunogenic peptide, that is capable of inducing an immune response. The immune response generated by the antigen may be B cell driven (antibody-mediated immune response) and/or T cell driven (cellular immune response).

### Fusion Protein

In the present context, the term “fusion protein” refers to peptides comprising a random order of two or more antigens from a pathogen or analogues thereof. The antigens may be fused together with or without an amino acid linker of varying length and sequence.

Fusion proteins may be produced by operatively linking two or more heterologous nucleic acid sequences encoding the amino acid sequences of the antigens of interest. To avoid protein aggregation in the down-stream production all cysteines in the fusion protein may be replaced with any amino acid, but serine is the preferred substitute because of its high structural similarity with cysteine.

The fusion proteins or antigens may comprise appropriate purification tags (or affinity tags) to allow purification from the crude biological source (e.g. recombinant expression system). Purification tags include, but are not limited to, His-tag, chitin binding protein (CBP), maltose binding protein (MBP) and glutathione-S-transferase (GST).

### Peptide

In the present context, the term “peptide” refers to a polymer composed of amino acid residues, related naturally occurring structural variants, and/or synthetic non-naturally occurring analogs thereof linked via peptide bonds. Conventional notation is used herein to portray peptide sequences: the left-hand end of a peptide sequence is the amino-terminus (N-terminus); the right-hand end of a peptide sequence is the carboxyl-terminus (C-terminus).

The peptide may be chemically modified by glycosylation, lipidation, prosthetic groups, or by containing additional amino acids such as e.g. a purification tag (e.g. his-tag) or a signal peptide. Purification tags are used to obtain highly pure protein preparations. The His-tag may comprise a methionine as the first amino acid followed by 6-8 histidines if used N-terminal, and 6-8 histidines followed by a STOP-codon if used C-terminal. When used N-terminal, the methionine start codon in the gene coding for the peptide fusion may be deleted to avoid false translational start sites.

Each peptide is encoded by a specific nucleic acid sequence. It will be understood that such sequences include analogues and variants thereof, wherein such nucleic acid sequences have been modified by substitution, insertion, addition or deletion of one or more nucleic acids. Substitutions are preferably conservative substitutions in the codon usage, which will not lead to any change in the amino acid sequence, but may be introduced to enhance the expression of the protein.

Peptides may be produced recombinantly or synthetically, for example, using an automated polypeptide synthesizer.

### Vaccine and Immunogenic Composition

In the present context, the terms “vaccine” and “immunogenic composition” refer to a composition comprising at least one antigen which is capable of providing active acquired immunity to pathogenic infection or disease. The “vaccine” or “immunogenic composition” may preferably comprise a fusion protein as described herein, which is capable of providing active acquired immunity to pathogenic infection or disease.

The vaccine or immunogenic composition may comprise an immunologically and pharmaceutically acceptable carrier or vehicle. Suitable carriers include, but are not limited to, polymers to which the peptide is bound by hydrophobic non-covalent interaction, such as a plastic, e.g. polystyrene, or polymers to which the peptide is covalently bound, such as a polysaccharide, or peptides, e.g. bovine serum albumin, ovalbumin or keyhole limpet haemocyanin. Suitable vehicles include, but are not limited to, diluents and suspending agents.

In the present context, the vaccine or immunogenic composition comprises the adjuvant composition of the present invention as well as an antigen. The antigen may be a fusion protein comprising several antigens or antigen fragments.

### DDA

One particular effective type of adjuvant that promotes a cell-mediated immune response is the class of quaternary ammonium compounds, such as the cationic surfactant dimethyldioctadecyl ammonium salt (DDA).

DDA is a synthetic amphiphilic compound comprising a hydrophilic cationic quaternary ammonium headgroup, and two hydrophobic saturated C18 alkyl chains. Thus, (DDA) is a lipophilic quaternary ammonium compound and has been used extensively as an adjuvant. In an aqueous environment, DDA molecules self-assemble to form vesicular bilayers similar to liposomes made from natural phospholipids.

The liposomal adjuvant composition according to the present invention comprises the cationic lipid DDA as various salts, most preferably dimethyldioctadecyl ammonium bromide or chloride (DDA-B or DDA-C) or the sulfate, phosphate or acetate salt hereof (DDA-X), or dimethyldioctadecenyl ammonium bromide or chloride (DODA-B or DODA-C) or the sulfate, phosphate or acetate compound hereof (DODA-X), which are pharmaceutically acceptable. Most preferably, the liposomal adjuvant composition according to the present invention comprises dimethyldioctadecyl ammonium bromide.

The CAS number of DDA is 3700-67-2.

However, the liposomal adjuvant composition according to the present invention can comprise further cationic lipids.

### MMG

Mycobacterial lipid monomycoloyl glycerol (MMG) is a glycolipid, which stabilizes the liposome formed with cationic surfactant DDA by incorporation into the liposome membrane.

The cationic liposomes are stabilized by incorporating glycolipids, such as MMG and optionally further glycolipids, into the liposome membranes.

Glycolipids like MMG have immunostimulatory properties themselves and can act in a synergistic way with the quaternary ammonium compounds (DDA) to enhance the immune response.

The synthetic analogue, referred to as MMG-1, consists of a hydrophilic glycerol head group and a lipid acid, displaying two hydrophobic saturated C14/C15 alkyl tails, linked via an ester bond. Furthermore, an array of MMG analogues, differing in the alkyl chain lengths (MMG-2; C16/C17, MMG-3; C10/C11, and MMG-4; C6/C7), or with respect to stereochemistry of head group (MMG-5; 2S) and lipid tail (MMG-6) exists.

MMG is preferably the synthetically manufactured glycolipid, MMG-1.

The chemical structure of the preferred MMG analogue is 3-hydroxy-2-tetradecyl-octadecanoic acid-2,3-dihydroxypropyl ester, preferably the (2R)-2,3-Dihydroxypropyl-3-hydroxy-2-tetradecyloctadecanoate diastereomer.

### CpG Deoxyoligonucleotides

CpG oligonucleotides (ODNs) are synthetic single-stranded CpG ODNs that contain unmethylated CpG dinucleotides in specific sequence contexts (CpG motifs).

These CpG motifs are present at a 20-fold greater frequency in bacterial DNA than in mammalian DNA. CpG ODNs activate Toll-like receptor 9 (TLR9), leading to strong immunostimulatory effects.

CpG ODN 2006 (ODN 7909) is a class B CpG ODN. Class B CpG ODNs preferably contain a full phosphorothioate backbone conferring nuclease resistance with one or more CpG dinucleotides. They strongly activate B cells but weakly stimulate IFNα secretion in pDCs.

### CpG ODN 2006 Sequence:

(24 mer; SEQ ID NO: 1)
5′-tcgtcgttttgtcgttttgtcgtt-3′

Bases are preferably phosphorothioate (nuclease resistant).

CpG ODN 1826 is a class B CpG ODN and is specific for TLR9.

### CpG ODN 1826 Sequence:

(20 mer; SEQ ID NO: 2)
5′-tccatgacgttcctgacgtt-3′.

### Poly (I:C)

The term “Poly (I:C)” or “Poly I:C” according to the present invention comprises single-stranded polyinosinic acid (Poly I) and single-stranded polycytidylic acid (Poly C) that are not associated by hydrogen bonding or covalent bonding at the time of administration as well as double-stranded or complexed Poly I/Poly C. Upon administration to a moist mucosal surface, uncomplexed Poly I and Poly C can form complexed Poly (I:C) and thus prime the innate immune system and provide protection against viral infection.

Preferably, Poly (I:C) is a synthetically manufactured double-stranded RNA analogue consisting of strands of polyinosinic acid annealed to strands of polycytidilic acid or analogues thereof. poly (A:U) (Polyadenylic-polyuridylic acid) could be used as an alternative analogue.

The molecular weight of Poly (I:C) depends on the polymer length. The Poly I:C potassium salt has a molecular weight specification of 10-750 kDa with a preferred range of 100-750 kDa. The CAS number of Poly I:C is 24939-03-5.

### Further Adjuvants

In the present context, the term “adjuvant” refers to a compound or mixture that further enhances the immune response. An adjuvant can serve as a tissue depot that slowly releases the antigen and as a lymphoid system activator, which non-specifically enhances the immune response, i.e. an immunomodulator.

By an immunomodulator is meant any component, which increases the effect of, directs, focuses, diversifies, accelerates or prolongs an immune response to a vaccine. This potentiation could be done un-specifically or specifically through pattern recognition receptors (PRRs) including but not limited to C-type lectin receptors (CLRs), Retinoic acid-Inducible Gene (RIG)-like receptors (RLRs), nucleotide-binding oligomerization domain (NOD)-like receptors and the toll-like receptors (TLRs).

The immunogenicity of the liposomes can be potentiated by inclusion of immunostimulating ligands (a.k.a. immunomodulators) for the so-called PRRs recognizing conserved molecular structures known as pathogen-associated molecular patterns (PAMPs) on pathogens. The ability of the PAMPs to modulate the innate immune response, and thereby the ensuing adaptive response, can with advantage be exploited for use in the prevention or treatment of pathogenic infection of the respiratory tract.

The PAMPs vary among the pathogens and include molecules such as cord factor (TDM), flagellin, lipopolysaccharide (LPS), peptidoglycans, and several nucleic acid variants, such as double-stranded ribonucleic acids (dsRNAs). Many immunomodulators inspired by the PAMPS have been developed over the years. These include Mincle agonists trehalose dibehenate (TDB) and synthetic monomycolyl glycerol (MMG), TLR4 agonist monophosphoryl lipid A (MPL), TLR3 agonist polyinosinic acid: polycytidylic acid (poly (I:C)), TLR 7/8 agonists like R848 and 3M052, TLR9 agonists like synthetic unmethylated CpG and Dectin-1 agonist Curdlan. The combination of liposomes with these PAMPs/immunomodulators is an attractive approach to develop a way of preventing or treating early pathogen infection, where the PAMPs/immunomodulators stimulate the antigen presenting cells, thereby potentiating the immune response.

### Infections

The term “infection” in the context of the present invention means an infection in the respiratory tract, such as the upper or lower respiratory tract, or a systemic infection caused by a pathogen, such as a virus, parasite or bacteria.

The viral infection may be a human coronavirus infection or an influenza A or B virus infection. Other viral infections may be caused by a picornavirus (e.g., rhinovirus), human parainfluenza virus, HIV, Zika, Ebola, Nipah, human Papilloma virus (HPV), human respiratory syncytial virus, adenovirus, enterovirus, or metapneumovirus, etc.

The bacterial infection may be caused by bacteria selected from but not limited to Chlamydia Sp, Streptococcus Sp, Haemophilus influenza, Moraxella catarrhalis, Burkholderia Sp., Pseudomonas Sp. and Mycobacterium Sp, etc.

The infection may also be caused by a parasite, which is the case for malaria. Malaria is an infectious disease transferred to humans through mosquitoes. The disease itself is caused by singled-celled parasites belonging to the plasmodium genus. The parasite causing the most severe cases of malaria is Plasmodium falciparum.

The “prevention” of a pathogenic infection, condition or disease refers to a vaccine or vaccine composition comprising the adjuvant composition of the present invention as well as an antigen that, in a statistical sample, reduces the occurrence of the infection, condition or disease in the treated subject relative to an untreated subject, or delays the onset or reduces the severity of one or more symptoms of the infection, condition or disease relative to the untreated control subject.

### Administration

The term “administration” in the context of the present invention means administration in various modes either by systemic administration, such as intramuscular, subcutaneous, intradermal or intraperitoneal injection or in delivery formulation or devices for e.g. topical-, intradermal-, intranasal-, sublingual-, oral- or pulmonary administration.

The adjuvant and/or vaccine composition according to the present invention is typically administered by systemic administration, more typically by subcutaneous or intramuscular in the range of once per two weeks, to once or twice per month, to once or twice per year.

### Subject

The term “subject” comprises humans of all ages, other primates (e.g., cynomolgus monkeys, rhesus monkeys); mammals in general, including mammals such as cattle, pigs, horses, sheep, goats, cats, and/or dogs; and/or birds. Preferred subjects are humans.

The term “subject” also includes healthy subjects of the population and, in particular, healthy subjects, who are exposed to pathogens and in need of protection against infection, such as health personal.

### Sequence Identity

In the present context, the term “sequence identity” refers to the sequence identity between genes or proteins at the nucleotide, base or amino acid level, respectively. Specifically, a DNA and a RNA sequence are considered identical if the transcript of the DNA sequence can be transcribed to the identical RNA sequence.

Thus, in the present context “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, word length=12, to obtain nucleotide sequences homologous to nucleic acid molecules of the invention. BLAST protein searches may be performed with the XBLAST program, score=50, word length=3 to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilizing the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

### DETAILED DESCRIPTION OF ASPECTS AND EMBODIMENTS OF THE INVENTION

The present invention will now be described in more detail in the following.

It should be noted that embodiments and features described in the context of one of the aspects of the present invention also apply to the other aspects of the invention.

All patent and non-patent references cited in the present application are hereby incorporated by reference in their entirety.

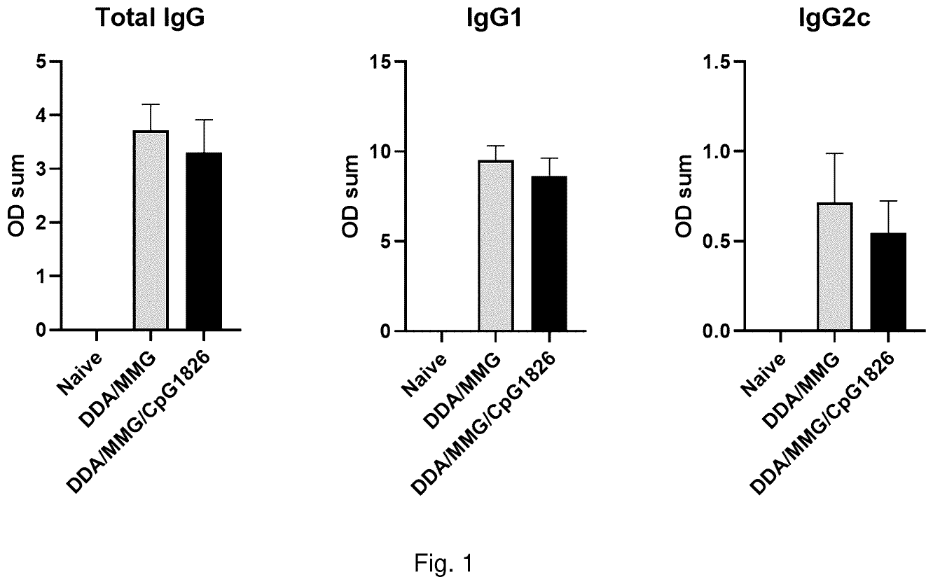

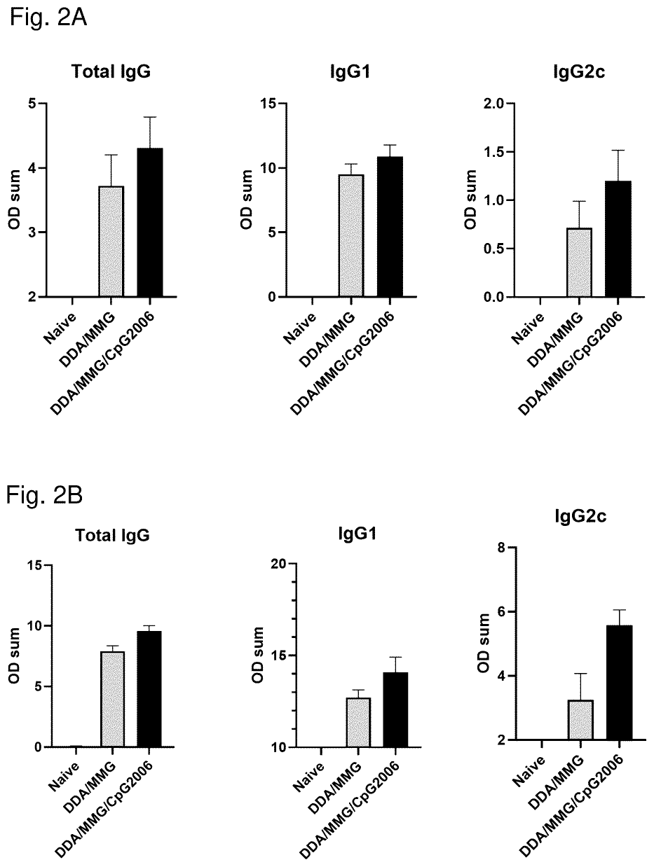

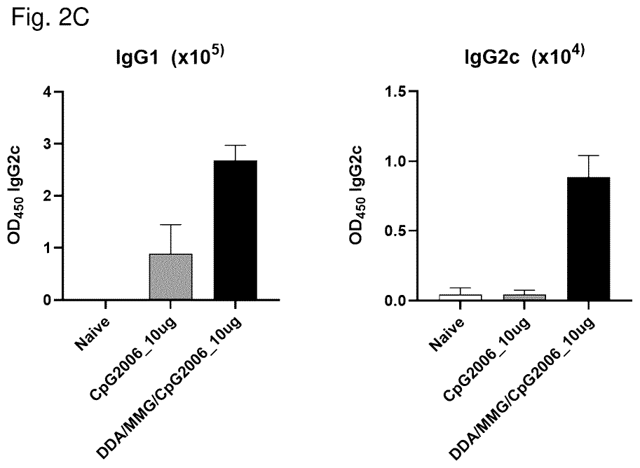

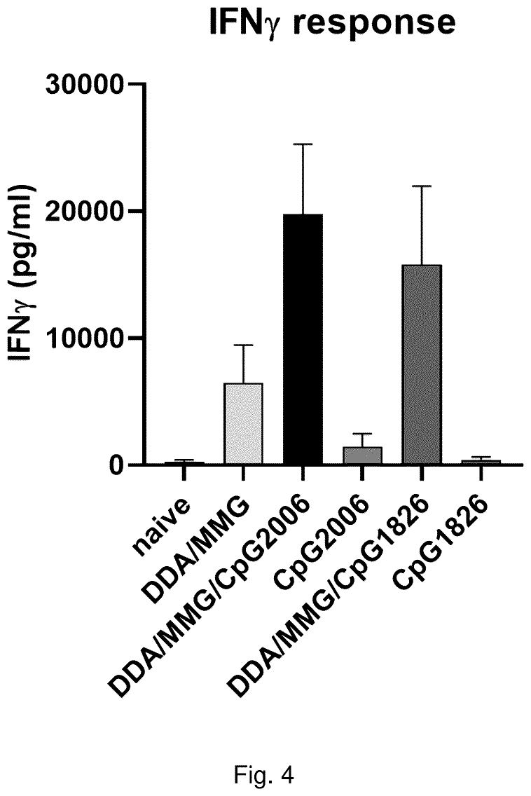

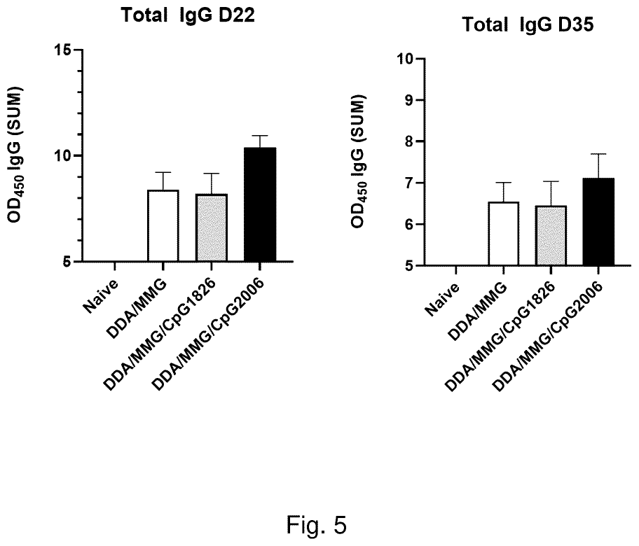

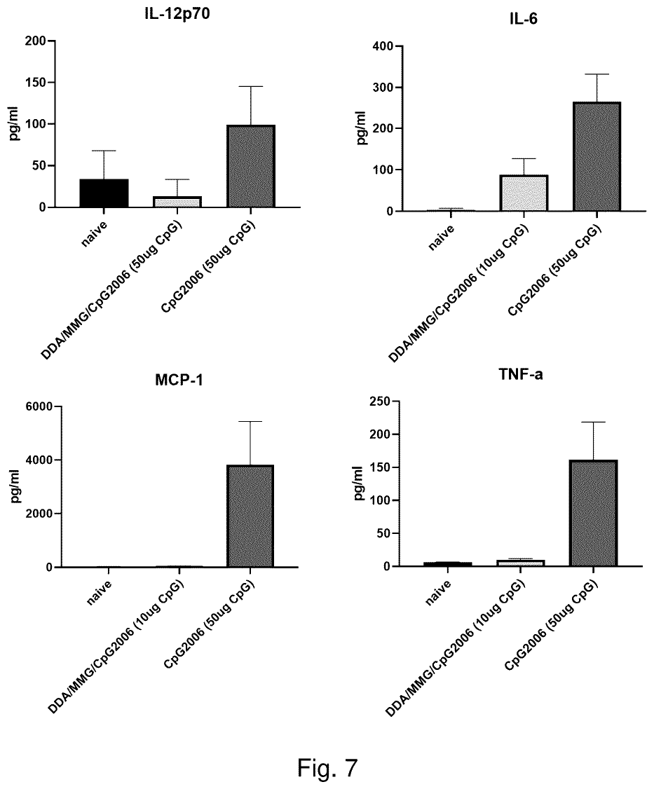

The present invention relates to an adjuvant composition—a part of the Cationic Adjuvant Formulation (CAF®) platform—called CAF®10b composed of DDA, MMG and CpG2006. The CAF® platform comprises different members of which the CAF®04 adjuvant composed of DDA and MMG, and the CAF®10 adjuvant composed of DDA, MMG and CpG1826, are of particular interest in relation to the present invention. The respective adjuvants all induce a strong T cell response although the encapsulation of a CpG specifically CpG1826 or CpG2006 in the DDA/MMG liposomes results in a superior T cell immune response compared to DDA/MMG alone (FIG. 4). Both CpG1826 and CpG2006 belong to a subclass of CpGs called “class B” known for activating B cells when used alone, i.e. inducing antibody responses or presenting antigens to T cells. The incorporation of CpG1826 into DDA/MMG liposomes does not increase antibody responses compared to DDA/MMG alone (FIG. 1), however, incorporating CpG2006 into DDA/MMG liposomes does result in an increased antibody response, specifically IgG1 and even more so IgG2c (FIG. 2A-B). This effect is not observed for CpG2006 alone indicating that CpG2006 acts synergistically with DDA/MMG liposomes to increase the antibody responses (FIG. 2C). With a high dose of CpG (50 μg), DDA/MMG/CpG2006 induced superior antibody responses compared to both DDA/MMG alone as well as DDA/MMG/CpG1826 at two time points (FIG. 5). Hence, both a cell-mediated immune response and a humoral immune response are obtained when combining DDA and MMG with CpG2006, which is not observed when combining DDA and MMG with another class B CpG, CpG1826. An increased humoral immune response (and in particular IgG2c) could potentially lead to a better prevention or treatment of an infectious disease since many disease-targets require strong antibody responses in addition to CMI responses for optimal immunity. Furthermore, the present invention reduces systemic inflammation associated with administering free CpG2006 thereby improving the safety profile (FIG. 7).

Thus, an aspect of the present invention relates to an adjuvant composition or liposomal adjuvant composition comprising or consisting of dimethyldioctadecyl ammonium salt (DDA), monomycoloyl glycerol (MMG), and the CpG ODN 2006 oligodeoxynucleotide having the sequence SEQ ID NO: 1 or a sequence having 90% identity to SEQ ID NO: 1.

One embodiment of the present invention relates to the adjuvant composition, wherein the composition comprises 1000-4000 μg/ml DDA, preferably wherein the composition comprises 2000-3000 μg/ml, more preferably 2500 μg/ml DDA.

A further embodiment of the present invention relates to the adjuvant composition, wherein the dimethyldioctadecyl ammonium (DDA) salt is selected from the group of salts comprising bromide, chloride, phosphate, acetate or other organic or inorganic salts, which are pharmaceutically acceptable, preferably the bromide salt.

Another embodiment of the present invention relates to the adjuvant composition, wherein the composition comprises 100-1000 μg/ml MMG, preferably wherein the composition comprises 300-700 μg/ml MMG, more preferably 500 μg/ml MMG.

A further embodiment of the present invention relates to the adjuvant composition, wherein the composition comprises 2500 μg/ml DDA and 500 μg/ml MMG.

Yet another embodiment of the present invention relates to the adjuvant composition, wherein the composition comprises the CpG ODN 2006 oligodeoxynucleotide having SEQ ID NO: 1 or a sequence having 90% identity to SEQ ID NO: 1 in an amount of 10-500 μg/ml, preferably 20-300 μg/ml, more preferably 50-200 μg/ml.

An additional embodiment of the present invention relates to the adjuvant composition, wherein the composition comprises 2500 μg/ml DDA and 500 μg/ml MMG, and 100 μg/ml CpG ODN 2006 oligodeoxynucleotide having SEQ ID NO: 1 or a sequence having 90% identity to SEQ ID NO: 1.

Another embodiment of the present invention relates to the adjuvant composition, wherein the CpG ODN 2006 oligodeoxynucleotide has a phosphorothioate backbone, i.e. single-stranded DNA oligonucleotide with a full phosphorothioate backbone.

An aspect of the present invention relates to a vaccine or vaccine composition comprising or consisting of the adjuvant composition of the present invention and at least one antigen.

One embodiment of the present invention relates to the vaccine or vaccine composition, wherein the vaccine composition is capable of efficiently inducing a cell mediated immune response and/or producing antigen-specific antibodies.

Another embodiment of the present invention relates to the vaccine or vaccine composition, wherein the antigen is an antigen from a pathogen causing an infectious disease.

A further embodiment of the present invention relates to the vaccine or vaccine composition, wherein the antigen is selected from the group comprising influenza antigens, coronavirus antigens, tuberculosis antigens, malaria antigens, or chlamydia antigens.

Yet another embodiment of the present invention relates to the vaccine or vaccine composition, wherein the tuberculosis antigen is a fusion protein selected from H56 having SEQ ID NO: 3, H107 having SEQ ID NO: 4, H107b having SEQ ID NO: 5, H107c having SEQ ID NO: 6 and H107e having SEQ ID NO: 7 or a fusion protein having 90% sequence identity to SEQ ID NO: 3, 4, 5, 6 or 7.

An additional embodiment of the present invention relates to the vaccine or vaccine composition, wherein the malaria antigen is a fusion protein comprising a antigenic domain Pro and I of fragment of Pfs230 and the antigenic domain 6C of Pfs48/45 having SEQ ID NO: 8 or a fusion protein having 90% sequence identity to SEQ ID NO: 8.

One embodiment of the present invention relates to the vaccine or vaccine composition, wherein the influenza antigen is a protein selected from the group comprising antigens or influenza A or B viruses, preferably a fusion protein of the influenza antigens HA, NA, NP, M1 and M2 having SEQ ID NO: 9 or a fusion protein having 90% sequence identity to SEQ ID NO: 9.

Another embodiment of the present invention relates to the vaccine or vaccine composition, wherein the Coronavirus antigen is a protein selected from the group comprising antigens of alpha, beta, gamma, or delta coronaviruses, preferably Spike from SARS COV-2 having SEQ ID NO: 10, Spike S1 from SARS COV-2 having SEQ ID NO: 11, or Spike S2 from SARS COV-2 having SEQ ID NO: 12 or a protein having 90% sequence identity to SEQ ID NO: 10, 11 or 12.

A further embodiment of the present invention relates to the vaccine or vaccine composition, wherein the Chlamydia antigen is the CTH522 antigen having SEQ ID NO: 13.

Another aspect of the present invention relates to the vaccine or vaccine composition according to the present invention for use in prevention or treatment of an infectious disease.

Yet another embodiment of the present invention relates to the vaccine or vaccine composition for use in prevention or treatment of tuberculosis.

An additional embodiment of the present invention relates to the vaccine or vaccine composition for use in prevention or treatment of malaria.

A further embodiment of the present invention relates to the vaccine or vaccine composition for use in prevention or treatment of influenza.

Another embodiment of the present invention relates to the vaccine or vaccine composition for use in prevention or treatment of coronavirus infection, preferably caused by SARS-COV-2.

Yet another embodiment of the present invention relates to the vaccine or vaccine composition for use according to the present invention, wherein the composition is administered to a subject by intradermal, intravenous, intramuscular or subcutaneous injection.

An additional embodiment of the present invention relates to the vaccine or vaccine composition for use according to the present invention, wherein the subject is selected from the group consisting of humans of all ages, other primates (e.g., cynomolgus monkeys, rhesus monkeys); mammals in general, including mammals, such as cattle, pigs, horses, sheep, goats, mink, ferrets, hamsters, cats and dogs, as well as birds.

A further embodiment of the present invention relates to the vaccine or vaccine composition for use according to the present invention, wherein the subject is a human.

Another embodiment of the present invention relates to the vaccine or vaccine composition for use according to the present invention, wherein the vaccine composition is administered as one dose. In another embodiment, the vaccine or vaccine composition is administered as at least two doses, such as at least three doses. The second or subsequent dose(s) may be administered as a booster dose at least one week, such as at least two weeks, such as at least three weeks or at least four weeks after the first dose.

The invention will now be described in further details in the following non-limiting examples.

### EXAMPLES

### Example 1—DDA/MMG Combined with a Class B CpG (CpG1826) does not Increase Antibody Responses

### Aim of Study

The aim of this study was to investigate whether a class B CpG, such as CpG1826, combined with DDA/MMG, would increase the antibody response as compared to using DDA/MMG alone.

### Material and Methods

Six to ten-week old female CB6F1 mice were immunized with 5 μg of H56 antigen (Aagaard, C. et al., 2011) formulated either in DDA/MMG (250/50 μg) or DDA/MMG/CpG1826 (250/50/10 μg), i.e. by using 100 μl of DDA/MMG (2500/500 μg/ml) or of DDA/MMG/CpG1826 (2500/500/100 μg/ml), respectively, in a total volume of 200 μL. Serum was collected 15 days after immunization and used to assess antigen-specific total IgG, IgG1 and IgG2c (n=10).

### Results

Based on previous work (e.g. Karlsen, K. et al. 2014), it can be hypothesized that DDA/MMG combined with a class B CpG, such as CpG1826, increases antibody responses compared to DDA/MMG alone, since class B CpGs are known to directly activate B cells and drive antibody responses. However, immunization with DDA/MMG/CpG1826 did not increase total IgG responses (nor IgG1/IgG2c) compared to DDA/MMG alone (FIG. 1).

### Conclusion

Class B CpGs, such as CpG1826, do not necessarily lead to increased antibody responses in DDA/MMG liposomes even though the CD4 T cell response is increased (see Karlsen, K. et al. 2014 and FIG. 4).

### Example 2—DDA/MMG Combined with CpG2006 Substantially Increases Antibody Responses

### Aim of Study

The aim of this study was to investigate whether CpG2006 combined with DDA/MMG could increase the antibody response when compared with DDA/MMG alone.

### Material and Methods

Six to ten-week old female CB6F1 mice were immunized with 5 μg of H56 antigen formulated either in a murine dose of DDA/MMG (250/50 μg) or a murine dose of DDA/MMG/CpG2006 (250/50/10 μg) and diluted to a total injection volume of 200 μL. Serum was collected 15 days later and used to assess antigen-specific total IgG, IgG1 and IgG2c by ELISA. At day 21, mice were boosted with a second dose of 5 μg of H56 antigen formulated either in DDA/MMG (250/50 μg) or DDA/MMG/CpG2006 (250/50/10 μg) in a total volume of 200 μL. Serum was collected one day later and used to assess antigen-specific total IgG, IgG1 and IgG2c by ELISA (n=10).

### Results

In contrast to immunization with DDA/MMG/CpG1826 (FIG. 1), incorporation of CpG2006 (another class B CpG) into DDA/MMG liposomes substantially increased the IgG responses compared to DDA/MMG liposomes alone, both at 15 days post immunization (FIG. 2A; Day 15) and one day after the 2nd immunization (FIG. 2B; Day 22). This counts for both IgG1 and IgG2 antibodies. Immunization with DDA/MMG/CpG2006 also displayed a substantially higher IgG1 and IgG2 antibody response than when using CpG2006 alone (FIG. 2C).

### Conclusion

To summarize, a combination of a class B CpG and DDA/MMG liposomes does not necessarily lead to increased antibodies, but a combination of DDA/MMG with CpG2006 surprisingly does. This effect is not observed for CpG2006 alone indicating that CpG2006 acts synergistically with DDA/MMG liposomes to increase the antibody responses.

### Example 3—DDA/MMG/CpG2006 Increases Antibody Responses Over DDA/MMG/CpG1826 with the H107e Antigen

### Aim of Study

Example 2 indicated that CpG2006 in combination with DDA/MMG could increase the antibody response, whereas the same effect was not observed for CpG1826 combined with DDA/MMG in example 1. The aim of this study was therefore to directly compare DDA/MMG/CpG2006 and DDA/MMG/CpG1826 in terms of their ability to induce IgG1 and IgG2c antibody responses. Said effect was also compared to naive, DDA/MMG, CpG2006 alone and CpG1826 alone.

### Material and Methods

Six to ten-week old female CB6F1 mice were immunized two times, three weeks apart with 5 μg of H107e antigen (Woodworth, J. S. et al, 2021) formulated in murine doses of either DDA/MMG (250/50 μg), DDA/MMG/CpG1826 (250/50/20 μg), DDA/MMG/CpG2006 (250/50/20 μg), CpG1826 (20 μg) or CpG2006 (20 μg) diluted to an injection volume of 200 μL. Serum was collected 1 day after the second immunization and used to assess antigen-specific IgG1 and IgG2a by ELISA (n=8). A ROUT outlier analysis with the strictest cut-off (Q=0.1%, prism) identified one outlier in the DDA/MMG/CpG1826 group that was removed from the dataset.

### Results

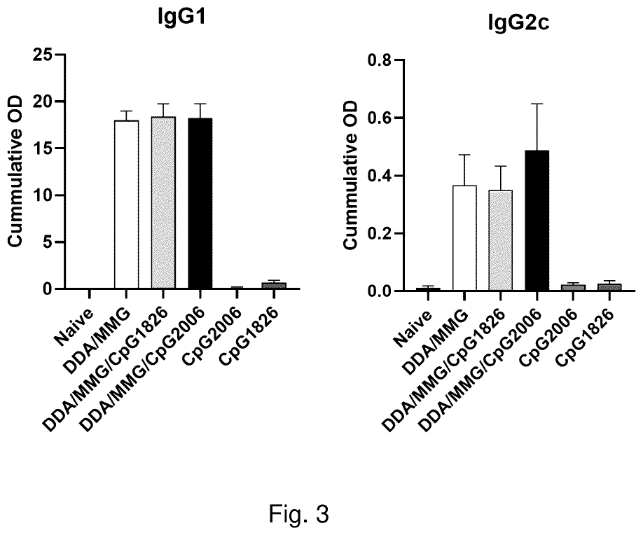

As observed in FIG. 1, immunization with DDA/MMG/CpG1826 did neither increase IgG1 nor IgG2c responses compared to DDA/MMG alone (FIG. 3). For IgG2c, in contrast, DDA/MMG/CpG2006 did increase the antibody response over both DDA/MMG and DDA/MMG/CpG1826 as well as CpG1826 and CpG2006 alone.

### Conclusion

DDA/MMG/2006 increases the IgG2c antibody response compared to DDA/MMG alone as well as DDA/MMG/CpG1826 with the H107e antigen. Furthermore, incorporation of CpGs into DDA/MMG increases the antibody responses compared to CpG1826 and CpG2006 alone.

### Example 4—DDA/MMG Combined with CpG2006 Significantly Increases T Cell Responses (Similarly to CpG1826)

### Aim of Study

The aim of this study was to investigate the effect of DDA/MMG combined with either CpG1826 or CpG2006 in terms of their ability to induce a T cell response (as measured by the IFNγ secretion).

### Material and Methods

Six to ten-week old female CB6F1 mice were immunized two times, three weeks apart with 5 μg of H56 antigen formulated in murine doses of either DDA/MMG (250/50 μg), DDA/MMG/CpG1826 (250/50/10 μg), DDA/MMG/CpG2006 (250/50/10 μg), CpG2006 (10 μg) or CpG1826 (10 μg) diluted to a total volume of 200 μL. Two weeks after 2nd immunization, splenocytes were isolated from 10 mice per group, and 2×10⁵ cells/well were stimulated in vitro with H56 protein for 3 days at 37° C. The accumulation of IFNγ in the cell cultures were measured by ELISA (n=9-10).

### Results

Immunization with DDA/MMG/CpG2006 increased the T cell response (measured by IFNγ secretion) compared to DDA/MMG and CpG2006 alone (FIG. 4). This was similar for DDA/MMG/CpG1826 as expected from Karlsen, K. et al., 2014.

### Conclusion

DDA/MMG/CpG2006 increases T cell responses compared to DDA/MMG alone, similarly to DDA/MMG/CpG1826.

### Example 5—DDA/MMG with 50 μg CpG2006 Increases Antibody Responses Over DDA/MMG with 50 μg CpG1826

### Aim of Study

The aim of this study was to investigate whether increasing the dose of the CpGs, namely CpG1826 and CpG2006, would affect their ability to increase the total IgG response when assessing the antigen-specific total IgG at two different time points.

### Material and Methods

Six to ten-week old female CB6F1 mice were immunized two times, three weeks apart with 5 μg of H56 antigen formulated in murine doses of either DDA/MMG (250/50 μg), DDA/MMG/CpG1826 (250/50/50 μg) or DDA/MMG/CpG2006 (250/50/50 μg) diluted to an injection volume of 200 μL. Serum was collected 1 and 14 days after the 2nd immunization and used to assess antigen-specific total IgG (n=7).

### Results

With a high dose of CpG (50 μg), DDA/MMG/CpG2006 induced superior antibody responses compared to both DDA/MMG alone as well as DDA/MMG/CpG1826 (FIG. 5). This was consistent across two time points; 1 day and 14 days after 2^{nd} immunization, i.e. day 22 and day 35, respectively (D22 and D35).

### Conclusion

DDA/MMG/CpG2006 induced superior antibody responses compared with DDA/MMG/CpG1826 at a higher dose of CpG (50 μg).

### Example 6—Incorporation of CpG2006 into DDA/MMG Liposomes Increases Antibody Responses Over a Range of Doses

### Aim of Study

Experiments in Example 2, 3 and 5 show that the addition of CpG2006 into DDA/MMG liposomes increases antibody responses in doses of 10, 20 and 50 μg, respectively. The aim of this study was therefore to investigate whether DDA/MMG combined with 2 μg CpG2006 would also have an effect on the antibody response and compare said response with DDA/MMG combined with 50 μg CpG2006.

### Material and Methods

Six to ten-week old female CB6F1 mice were immunized two times, three weeks apart with 5 μg of H56 antigen formulated either in murine doses of DDA/MMG (250/50 μg) liposomes with 2 μg or 50 μg CpG2006 corresponding to 2500/500 μg/ml DDA/MMG and 20-500 μg/ml CpG2006 and diluted to an injection volume of 200 μL. Serum was collected 1 day after the 2nd immunization and used to assess antigen-specific total IgG, IgG1 and IgG2c (n=7-8).

### Results

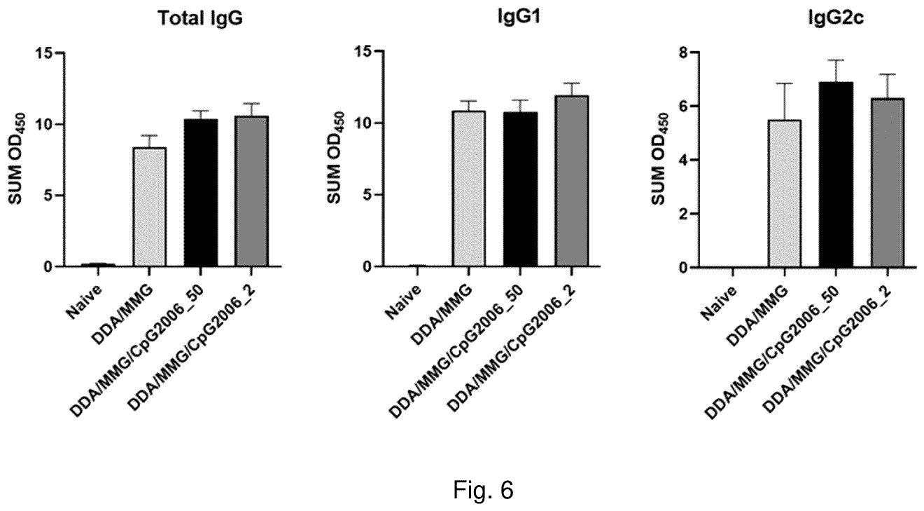

Experiments in Example 2, 3 and 5 show that the addition of CpG2006 into DDA/MMG liposomes increases antibody responses in doses of 10, 20 and 50 μg. In this experiment, the inventors demonstrate that immunization with DDA/MMG combined with 2 μg CpG2006 also increases the antibody response (IgG2c, FIG. 6, right) compared to DDA/MMG. Increased antibody responses with DDA/MMG combined with 50 μg CpG2006 were previously confirmed as observed in FIG. 5.

### Conclusion

DDA/MMG/CpG2006 induce superior antibody responses compared to DDA/MMG in doses ranging from 2-50 μg CpG2006.

### Example 7—Combining CpG2006 with DDA/MMG Reduces Systemic Inflammation

### Aim of Study

The aim of this study was to investigate whether administered CpG2006 in combination with DDA/MMG would affect systemic inflammation as measured by the IL12p70, IL-6, MCP-1 and TNF-α secretion.

### Material and Methods

Six to ten-week old female CB6F1 mice were immunized with 5 μg of H56 antigen formulated either in murine doses of DDA/MMG/CpG2006 (250/50/50 μg) or CpG2006 alone (50 μg) diluted to an injection volume of 200 μL. Serum was collected two days later and used to assess IL12p70, IL-6, MCP-1 and TNF-a by MSD (n=4).

### Results

Experiments in Example 2 show that combination of CpG2006 with DDA/MMG liposomes increases antibody responses compared to using DDA/MMG alone or CpG2006 alone. This experiment shows that combination of CpG2006 with DDA/MMG liposomes (DDA/MMG/CpG2006) reduces the systemic inflammation associated with administering free CpG2006 (FIG. 7).

### Conclusion

To summarize, combination of CpG2006 with DDA/MMG liposomes reduces systemic inflammation which could be linked to less side effects.

### Example 8—DDA/MMG Liposomes are Superior to DOTAP-Liposomes for Inducing Antibody Responses with CpG2006

### Aim of Study

It is evident from the previous examples that CpG2006 combined with DDA/MMG is capable of inducing both humoral and T-cell responses. The aim of this study was therefore to test whether exchanging the DDA/MMG components of the liposomes with DOTAP/DC-Chol would affect the antibody responses observed when administering DDA/MMG/CpG2006 liposomes formulated with the H56 antigen.

### Material and Methods

Six to ten-week old female CB6F1 mice were immunized twice, three weeks apart with 5 μg of H56 antigen formulated in murine doses of either DDA/MMG/CpG2006 (250/50/10 μg) or DOTAP/DC-chol/CpG2006 (150/150/10 μg) diluted to an injection volume of 200 μl. Serum was collected two weeks later and used to assess antigen-specific IgG1 and IgG2c by ELISA (n=4).

### Results

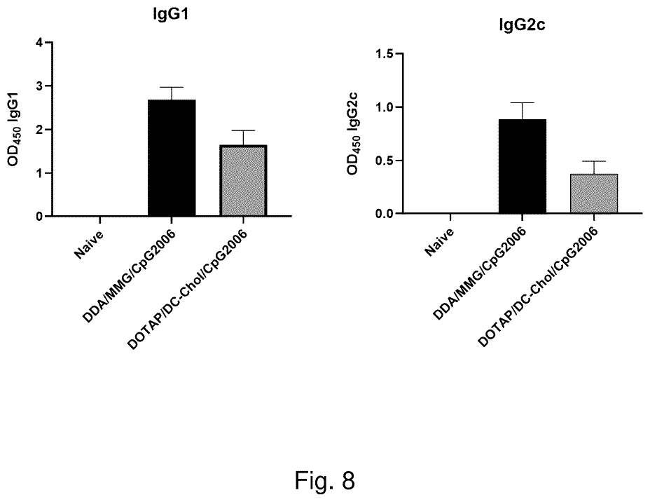

Experiments in Example 2 show that combining CpG2006 with DDA/MMG liposomes increases antibody responses compared to using DDA/MMG alone or CpG2006 alone. This experiment shows that combining CpG2006 with DDA/MMG liposomes (DDA/MMG/CpG2006) gives superior antibody responses compared to combining CpG2006 with another cationic liposome formulation (DOTAP/DC-chol/CpG2006) (FIG. 8).

### Conclusion

DDA/MMG/CpG2006 is superior to DOTAP/DC-chol/CpG2006 for inducing antibody responses.

### Example 9—DDA/MMG/CpG2006 Liposomes Induce Robust Antibody Responses in Non-Human Primates

### Aim of Study

The aim of this study was to test whether H107 antigen formulated in DDA/MMG/CpG2006 could increase the IgG response in non-human primates as compared to a control and H107 antigen formulated in DDA/MMG/poly (IC).

### Material and Methods

Cynomolgus macaques were immunized two times, four weeks apart with 20 μg of H107 antigen formulated in NHP doses of either DDA/MMG/poly (I:C) (625/125/30 μg) or DDA/MMG/CpG2006 (625/125/50 μg). Serum was collected at 0, 2, 4, 6, 8, 10, 12 and 30 weeks after immunization and used to assess antigen-specific IgG by ELISA (n=5).

### Results

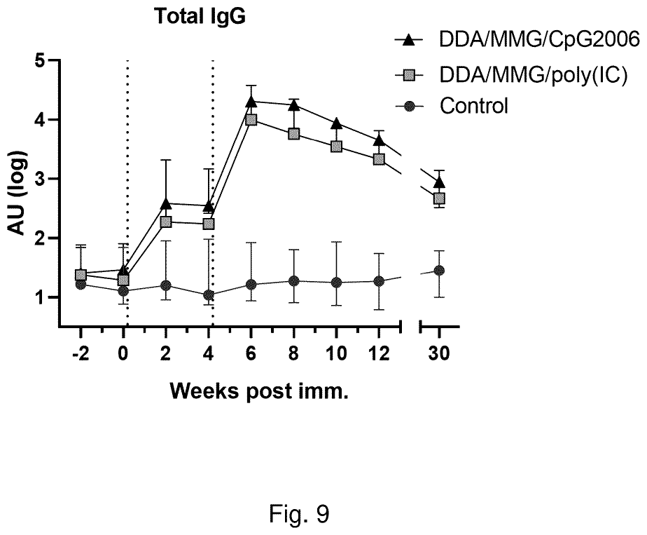

In a highly relevant species (NHPs), this experiment shows that DDA/MMG/CpG2006 induces an increased antibody response compared to a state-of-the-art adjuvant, CAF®09b (Mørk S. K. et al. 2022), consisting of DDA/MMG/Poly (I:C) (FIG. 9).

### Conclusion

To summarize, DDA/MMG/CpG2006 is a novel adjuvant with a potent adjuvanticity in a relevant target species.

### Example 10—DDA/MMG with 20 μg CpG2006 Increases Th17 Responses and Protection Against Mycobacterium tuberculosis Infection Compared to DDA/MMG with 20 μg CpG1826

### Aim of Study

The other examples have demonstrated that different antigens (H107, H56) formulated in DDA/MMG/CpG2006 could increase both the IgG, in particular the IgG2c, and the T-cell responses as compared to DDA/MMG. Thus, the aim of this study was to investigate whether DDA/MMG/CpG2006 could be used as a vaccine adjuvant and protect mice challenged with Mycobacterium tuberculosis. In addition, Th17 cells have been implicated in protection against major pathogens such as influenza, chlamydia, Klebsiella pneumoniae, group A streptococci and tuberculosis. Hence, the inventors also wanted to measure the IL-17A response as an indicator of the Th17 cells to investigate whether the DDA/MMG/CpG2006 vaccine adjuvant increases Th17 responses.

### Material and Methods

Six to ten-week old female CB6F1 mice were immunized two times, three weeks apart with 1 μg of H107 antigen formulated in murine doses of either DDA/MMG (250/50 μg), DDA/MMG/CpG1826 (250/50/20 μg) or DDA/MMG/CpG2006 (250/50/20 μg) diluted to an injection volume of 200 μL. Two weeks after the second immunization, Th17 responses were analysed by an IL-17A ELISA on culture supernatants from splenocytes stimulated with H107. Six weeks after the second immunization, all animals were aerosol challenged (20-50 CFU) with the Mycobacterium tuberculosis strain Erdman. Four weeks later, the number of mycobacteria was determined in individual lungs by plating of serial dilution of lung homogenate.

### Results

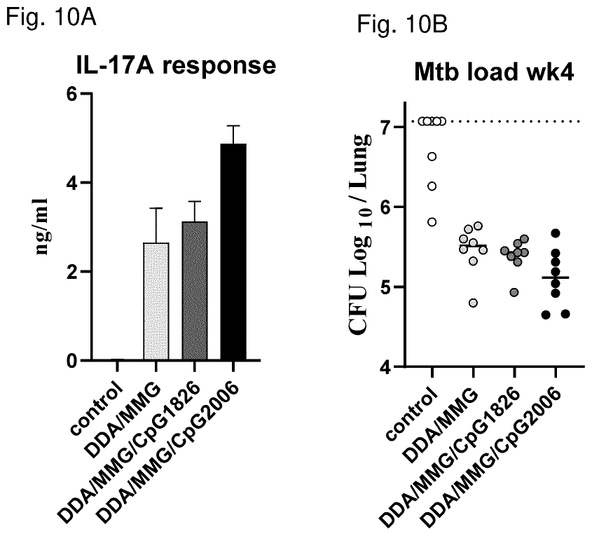

Two weeks after the last immunization, all groups induced a Th17 response but the highest response was seen for the group immunized with H107 in DDA/MMG/CpG2006 (FIG. 10A). The groups immunized with H107 in DDA/MMG and DDA/MMG/CpG1826 were similar. After Mycobacterium tuberculosis challenge, all vaccine groups had lower bacterial burdens compared to saline injected control animals (FIG. 10B). The animals immunized with H107 in DDA/MMG/CpG2006 had the lowest bacterial burden, showing that DDA/MMG/CpG2006 induce improved protection compared to DDA/MMG/CpG1826.

### Conclusion

DDA/MMG/CpG2006 increase Th17 responses and induce superior protection against respiratory infection with Mycobacterium tuberculosis compared to DDA/MMG/CpG1826.

### Example 11-DDA/MMG/CpG2006 Liposomes Induce Long-Lived Protection Against Genital Chlamydia Infection

### Aim of Study

The aim of this study was to test whether CTH522 antigen formulated in DDA/MMG/CpG2006 could induce a protection against Chlamydia trachomatis infection. The antibody response was also assessed at six weeks and 56 weeks following the vaccine administration. The antibody response and protection against Chlamydia trachomatis infection was also tested for the state-of-the-art adjuvant, CAF®09b.

### Material and Methods

Six to ten-week old female CB6F1 mice were immunized three times, two weeks apart with 10 μg of CTH522 antigen formulated in murine doses of either DDA/MMG/CpG2006 (250/50/20 μg) or DDA/MMG/Poly (I:C) (250/50/15 μg) diluted to an injection volume of 200 μL. Antibody responses against CTH522 were measured in serum by IgG ELISA at week 6 and 56. At week 58, animals received an intravaginal infection with 1×10⁵ IFU Chlamydia trachomatis Serovar D/UW-3/Cx and bacterial load was determined in individual vaginal swabs at day 3, 7 and 10 post infection.

### Results

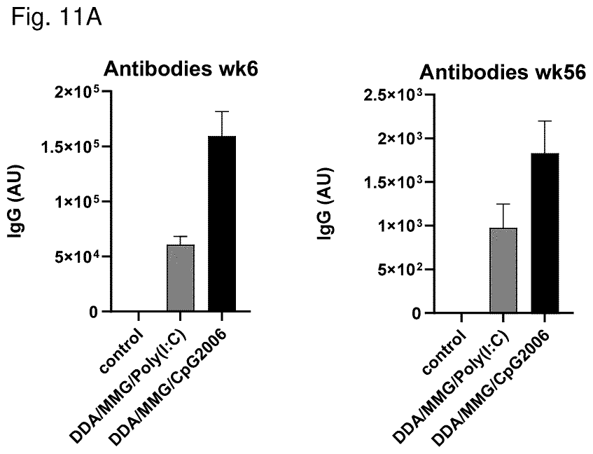

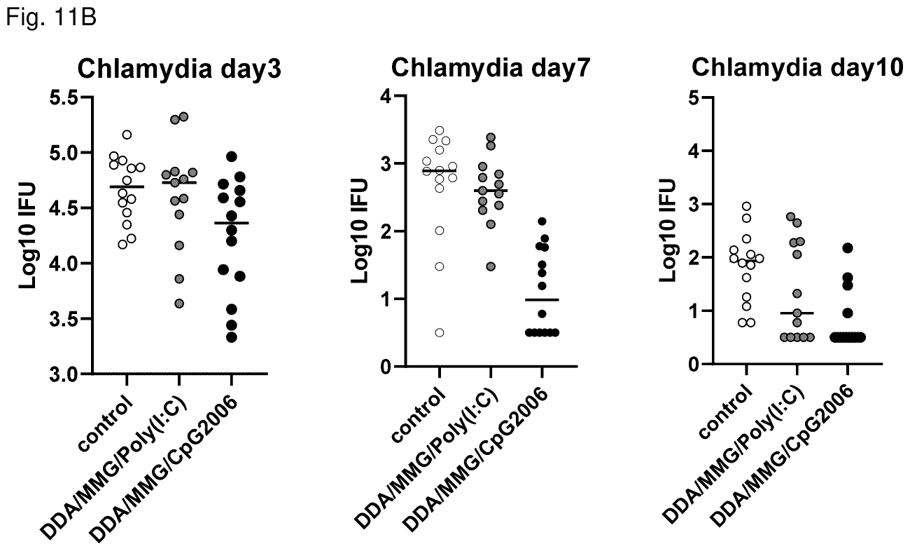

This experiment shows that DDA/MMG/CpG2006 induces an increased antibody response compared to a state-of-the-art adjuvant, CAF®09b (Mørk S. K. et al. 2022), consisting of DDA/MMG/Poly (I:C) using the CTH522 antigen (FIG. 11). This response is long-lived and was detectable 56 weeks after vaccination (FIG. 11A). After chlamydia challenge, animals vaccinated with DDA/MMG/CpG2006 had lower bacterial burdens in vaginal swabs and cleared the chlamydia infection faster than animals vaccinated with DDA/MMG/Poly (I:C) (FIG. 11B).

### Conclusion

DDA/MMG/CpG2006 induce robust and long-lived protection against Chlamydia trachomatis infection.

### Example 12—DDA/MMG/CpG2006 Increases Antibody and Th17 Responses Over DDA/MMG/CpG1826 with the Influenza Hemagglutinin (HA) Antigen

### Aim of Study

As demonstrated in example 10 and 11, the DDA/MMG/CpG2006 vaccine adjuvant can be used to induce protection against Mycobacterium tuberculosis infections and genital chlamydia infections, respectively. Next, the inventors wanted to investigate whether DDA/MMG/CpG2006 could also be used as a vaccine adjuvant for influenza. The aim of this study was therefore to measure the antibody responses and Th17 responses following immunization with the HA antigen formulated in either DDA/MMG/CpG2006 or DDA/MMG/CpG1826.

### Materials and Methods

Six to ten-week old female CB6F1 mice were immunized two times, three weeks apart with 5 μg of HA antigen (Influenza A H1N1 (A/Puerto Rico/8/1934)) (SEQ ID NO: 14) formulated in murine doses of either DDA/MMG/CpG1826 (250/50/20 μg) or DDA/MMG/CpG2006 (250/50/20 μg) diluted to an injection volume of 200 μL. Serum was collected 1 day after the second immunization and used to assess antigen-specific total IgG, IgG1 and IgG2a by ELISA (n=6). Splenocytes were collected 14 days after the second immunization and used to assess Th17 responses after re-stimulation with the HA antigen for 72 hours (n=6).

### Results

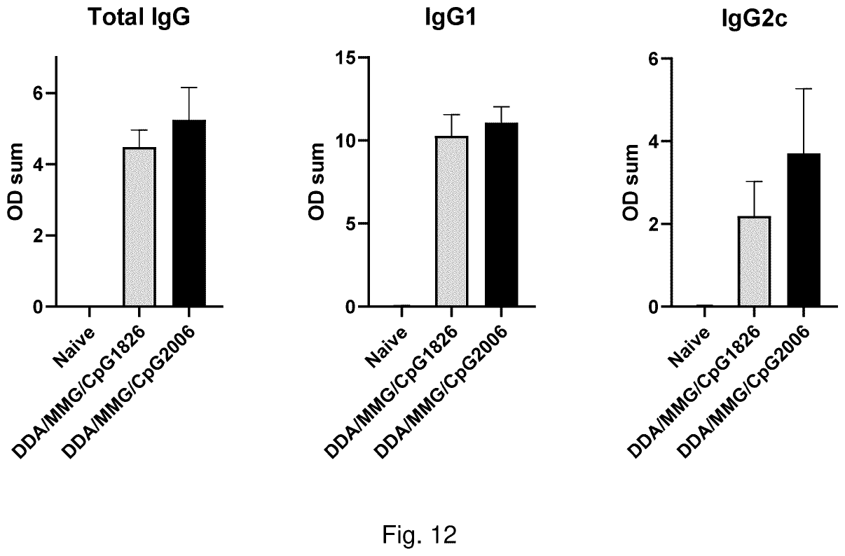

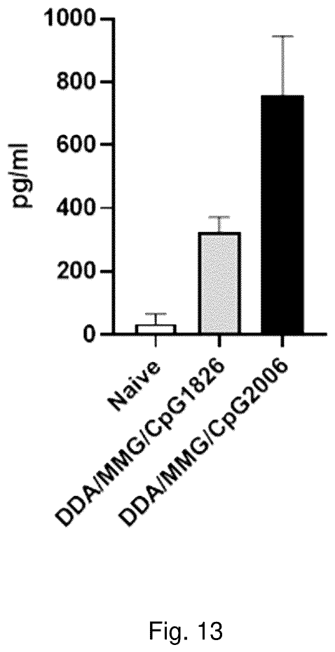

DDA/MMG/CpG2006 increased the antibody responses for total IgG and IgG2c over DDA/MMG/CpG1826, whereas the IgG1 response was comparable to that obtained using DDA/MMG/CpG1826 (FIG. 12). The IL-17A response, indicative of a Th17 response, was also increased when DDA/MMG/CpG2006 was used as the vaccine adjuvant as compared to the DDA/MMG/CpG1826 vaccine adjuvant (FIG. 13).

### Conclusion

DDA/MMG/CpG2006 increases the overall IgG and IgG2c antibody responses and the Th17 responses compared to DDA/MMG/CpG1826 with the HA antigen. Hence, the DDA/MMG/CpG2006 adjuvant composition can be used as a vaccine adjuvant in the treatment of influenza and is more immunogenic than DDA/MMG/CpG1826.

### REFERENCES

WO2010006607 A2
WO2006136162 A2
WO2014063704 A2
WO2015161853 A1
Aagaard, C. et al. 2011, “A multistage tuberculosis vaccine that confers efficient protection before and after exposure”, Nat Med, 17, 189-194
Karlsen, K. et al. 2014, “A stable nanoparticulate DDA/MMG formulation acts synergistically with CpG ODN 1826 to enhance the CD4⁺ T-cell response”, Nanomedicine, vol. 9, No. 17
Seder et al. (2008), Nat. Rev. Immunol. 8, 247-258
Woodworth, J. S., et al. 2021, “A Mycobacterium tuberculosis-specific subunit vaccine that provides synergistic immunity upon co-administration with Bacillus Calmette-Guérin”, Nat Commun. vol 12, No 6658
Mørk, S. K. et al. 2022, “Personalized therapy with peptide-based neoantigen vaccine (EVX-01) including a novel adjuvant, CAF®09b, in patients with metastatic melanoma”, Oncoimmunology, 2022 Jan. 10; 11 (1): 2023255.

Sequence listing
(nucleotide sequence of CpG ODN 2006)
SEQ ID NO: 1
5′-tcgtcgttttgtcgttttgtcgtt-3′ (24 mer; single-stranded)
(nucleotide sequence of CpG ODN 1826)
SEQ ID NO: 2
5′-tccatgacgttcctgacgtt-3′ (20 mer; single-stranded)
(amino acid sequence for TB antigen fusion H56)
SEQ ID NO: 3
MSRPGLPVEYLQVPSPSMGRDIKVQFQSGGNNSPAVYLLDGLRAQDDYNGWDINTPAFEW
YYQSGLSIVMPVGGQSSFYSDWYSPACGKAGCQTYKWETFLTSELPQWLSANRAVKPTGS
AAIGLSMAGSSAMILAAYHPQQFIYAGSLSALLDPSQGMGPSLIGLAMGDAGGYKAADMW
GPSSDPAWERNDPTQQIPKLVANNTRLWVYCGNGTPNELGGANIPAEFLENFVRSSNLKFQ
DAYNAAGGHNAVFNFPPNGTHSWEYWGAQLNAMKGDLQSSLGAGTEQQWNFAGIEAAAS
AIQGNVTSIHSLLDEGKQSLTKLAAAWGGSGSEAYQGVQQKWDATATELNNALQNLARTI
SEAGQAMASTEGNVTGMFAVIAGVDQALAATGQASQRAAGASGGVTVGVGVGTEQRNLS
VVAPSQFTFSSRSPDFVDETAGQSWCAILGLNQFH
(amino acid sequence for TB antigen fusion H107)
SEQ ID NO: 4
MQAETAVNTLFEKLEPMASILDPGASQSTTNPIFGMPSPGSSTPVGQLPPAATQTLGQLGEM
SGPMGGSGNPADEEAAQMGLLGTSPLSNHPLAGGSGPSAGAGLLRAESLPGAGGSLTRTPL
MSQLIEKPVATEQQWNFAGIEAAASAIQGNVTSIHSLLDEGKQSLTKLAAAWGGSGSEAYQ
GVQQKWDATATELNNALQNLARTISEAGQAMASTEGNVTGMFAAADYDKLFRPHEGMEAP
DDMAAQPFFDPSASFPPAPASANLPKPNGQTPPPTSDDLSERFVSAPPPPPPPPPPPPPTPMPI
AAGEPPSPEPAASKPPTPPMPIAGPEPAPPKPPTPPMPIAGPEPAPPKPPTPPMPIAGPAPTPTE
SQLAPPRPPTPQTPTGAPQQPESPAPHVPSHGPHQPRRTAPAPPWAKMPIGEPPPAPSRPSA
SPAEPPTRPAPQHSRRARRGHRYRTDTERNVGKVATGPSIQARLRAEEASGAQLAPGTEPSP
APLGQPRSYLAPPTRPAPTEPPPSPSPQRNSGRRAERRVHPDLAAQHAAAQPDSITAATTGG
RRRKRAAPDLDATQKSLRPAAKGPKVKKVKPQKPKATKPPKVVSQRGWRHWVHALTRINL
GLSPDEKYELDLHARVRRNPRGSYQIAVVGLQGGAGKTTLTAALGSTLAQVRADRILALDAD
PGAGNLADRVGRQSGATIADVLAEKELSHYNDIRAHTSVNAVNLEVLPAPEYSSAQRALSDA
DWHFIADPASRFYNLVLADSGAGFFDPLTRGVLSTVSGVVVVASVSIDGAQQASVALDWLR
NNGYQDLASRASVVINHIMPGEPNVAVKDLVRHFEQQVQPGRVVVMPWDRHIAAGTEISL
DLLDPIYKRKVLELAAALSDDFERAGRRTEQQWNFAGIEAAASAIQGNVTSIHSLLDEGKQS
LTKLAAAWGGSGSEAYQGVQQKWDATATELNNALQNLARTISEAGQAMASTEGNVTGMF
ATENLTVQPERLGVLASHHDNAAVDASSGVEAAAGLGESVAITHGPYSSQFNDTTEQQWN
FAGIEAAASAIQGNVTSIHSLLDEGKQSLTKLAAAWGGSGSEAYQGVQQKWDATATELNN
ALQNLARTISEAGQAMASTEGNVTGMFASRAFIIDPTISAIDGLYDLLGIGIPNQGGILYSSLE
YFEKALEELAAAFPGDGWLGSAADKYAGKNRNHVNFFQELADLDRQLISLIHDQANAVQTT
RDILEGAKKGLEFVRPVAVDLTYIPVVGHALSAKTLINATQLLKLLAKLAELVAAAIADIISDVA
DIIKGTLGEVWEFITNALNGLKELWDKLTGWVTGLFSRGWSNLESFFAGVPGLTGATSGLS
QVTGLFGAAGLSASSGLAHADSLASSASLPALAGIGGGSGFGGLPSLAQVHAASTRQALRP
RADGPVGAAAEQVGGQSQLVSAQGSQGMGGPVGMGGMHPSSGASKGTTTKKYSEGAAA
GTEDAERAPVEADAGGGQKVLVRNVVTEQQWNFAGIEAAASAIQGNVTSIHSLLDEGKQSL
TKLAAAWGGSGSEAYQGVQQKWDATATELNNALQNLARTISEAGQAMASTEGNVTGMFA
APKTYSEELKGTDTGQASQIQMSDPAYNINISLPSYYPDQKSLENYIAQTRDKFLSAATSSTP
REAPYELNITSATYQSAIPPRGTQAVVLKVYQNAGGTHPTTTYKAFDWDQAYRKPITYDTLW
QADTDPLPVVFPIVQGELSKQTGQQVSIAPNAGLDPVNYQNFAVTNDGVIFFFNPGELLPEA
AGPTQVLVPRSAIDSMLAGDLVGPGSAEYAAANPTGPASVQGMSQDPVAVAASNNPELTTL
TAALSGQLNPQVNLVDTLNSGQYTVFAPTNAAFSKLPASTIDELKTNSSLLTSILTYHVVAGQ
TSPANVVGTRQTLQGASVTVTGQGNSLKVGNADVVSGGVSTANATVYMIDSVLMPPAVSQ
DTSPKPATSPAAPVTTAAMADPAADLIGRGSAQYAAQNPTGPGSVAGMAQDPVATAASNNP
MLSTLTSALSGKLNPDVNLVDTLNGGEYTVFAPTNAAFDKLPAATIDQLKTDAKLLSSILTYH
VIAGQASPSRIDGTHQTLQGADLTVIGARDDLMVNNAGLVSGGVHTANATVYMIDTVLMPP
AQ
(amino acid sequence for TB antigen fusion H107b)
SEQ ID NO: 5
MTEQQWNFAGIEAAASAIQGNVTSIHSLLDEGKQSLTKLAAAWGGSGSEAYQGVQQKWD
ATATELNNALQNLARTISEAGQAMASTEGNVTGMFAQAETAVNTLFEKLEPMASILDPGASQ
STTNPIFGMPSPGSSTPVGQLPPAATQTLGQLGEMSGPMGGSGNPADEEAAQMGLLGTSPL
SNHPLAGGSGPSAGAGLLRAESLPGAGGSLTRTPLMSQLIEKPVATEQQWNFAGIEAAASAI
QGNVTSIHSLLDEGKQSLTKLAAAWGGSGSEAYQGVQQKWDATATELNNALQNLARTISE
AGQAMASTEGNVTGMFAAADYDKLFRPHEGMEAPDDMAAQPFFDPSASFPPAPASANLPKP
NGQTPPPTSDDLSERFVSAPPPPPPPPPPPPPTPMPIAAGEPPSPEPAASKPPTPPMPIAGPEP
APPKPPTPPMPIAGPEPAPPKPPTPPMPIAGPAPTPTESQLAPPRPPTPQTPTGAPQQPESPAP
HVPSHGPHQPRRTAPAPPWAKMPIGEPPPAPSRPSASPAEPPTRPAPQHSRRARRGHRYRT
DTERNVGKVATGPSIQARLRAEEASGAQLAPGTEPSPAPLGQPRSYLAPPTRPAPTEPPPSPS
PQRNSGRRAERRVHPDLAAQHAAAQPDSITAATTGGRRRKRAAPDLDATQKSLRPAAKGPK
VKKVKPQKPKATKPPKVVSQRGWRHWVHALTRINLGLSPDEKYELDLHARVRRNPRGSYQI
AVVGLQGGAGKTTLTAALGSTLAQVRADRILALDADPGAGNLADRVGRQSGATIADVLAEK
ELSHYNDIRAHTSVNAVNLEVLPAPEYSSAQRALSDADWHFIADPASRFYNLVLADSGAGFF
DPLTRGVLSTVSGVVVVASVSIDGAQQASVALDWLRNNGYQDLASRASVVINHIMPGEPNV
AVKDLVRHFEQQVQPGRVVVMPWDRHIAAGTEISLDLLDPIYKRKVLELAAALSDDFERAGR
RTEQQWNFAGIEAAASAIQGNVTSIHSLLDEGKQSLTKLAAAWGGSGSEAYQGVQQKWD
ATATELNNALQNLARTISEAGQAMASTEGNVTGMFASRAFIIDPTISAIDGLYDLLGIGIPNQ
GGILYSSLEYFEKALEELAAAFPGDGWLGSAADKYAGKNRNHVNFFQELADLDRQLISLIHD
QANAVQTTRDILEGAKKGLEFVRPVAVDLTYIPVVGHALSAKTLINATQLLKLLAKLAELVAAA
IADIISDVADIIKGTLGEVWEFITNALNGLKELWDKLTGWVTGLFSRGWSNLESFFAGVPGL
TGATSGLSQVTGLFGAAGLSASSGLAHADSLASSASLPALAGIGGGSGFGGLPSLAQVHAA
STRQALRPRADGPVGAAAEQVGGQSQLVSAQGSQGMGGPVGMGGMHPSSGASKGTTTK
KYSEGAAAGTEDAERAPVEADAGGGQKVLVRNVVTEQQWNFAGIEAAASAIQGNVTSIHSL
LDEGKQSLTKLAAAWGGSGSEAYQGVQQKWDATATELNNALQNLARTISEAGQAMASTEG
NVTGMFAAPKTYSEELKGTDTGQASQIQMSDPAYNINISLPSYYPDQKSLENYIAQTRDKFL
SAATSSTPREAPYELNITSATYQSAIPPRGTQAVVLKVYQNAGGTHPTTTYKAFDWDQAYRK
PITYDTLWQADTDPLPVVFPIVQGELSKQTGQQVSIAPNAGLDPVNYQNFAVTNDGVIFFFN
PGELLPEAAGPTQVLVPRSAIDSMLAGDLVGPGSAEYAAANPTGPASVQGMSQDPVAVAAS
NNPELTTLTAALSGQLNPQVNLVDTLNSGQYTVFAPTNAAFSKLPASTIDELKTNSSLLTSILT
YHVVAGQTSPANVVGTRQTLQGASVTVTGQGNSLKVGNADVVSGGVSTANATVYMIDSVL
MPPAVSQDTSPKPATSPAAPVTTAAMADPAADLIGRGSAQYAAQNPTGPGSVAGMAQDPV
ATAASNNPMLSTLTSALSGKLNPDVNLVDTLNGGEYTVFAPTNAAFDKLPAATIDQLKTDAK
LLSSILTYHVIAGQASPSRIDGTHQTLQGADLTVIGARDDLMVNNAGLVSGGVHTANATVY
MIDTVLMPPAQ
(amino acid sequence for TB antigen fusion H107c)
SEQ ID NO: 6
MQAETAVNTLFEKLEPMASILDPGASQSTTNPIFGMPSPGSSTPVGQLPPAATQTLGQLGEM
SGPMGGSGNPADEEAAQMGLLGTSPLSNHPLAGGSGPSAGAGLLRAESLPGAGGSLTRTPL
MSQLIEKPVATEQQWNFAGIEAAASAIQGNVTSIHSLLDEGKQSLTKLAAAWGGSGSEAYQ
GVQQKWDATATELNNALQNLARTISEAGQAMASTEGNVTGMFAWVHALTRINLGLSPDEK
YELDLHARVRRNPRGSYQIAVVGLQGGAGKTTLTAALGSTLAQVRADRILALDADPGAGNLA
DRVGRQSGATIADVLAEKELSHYNDIRAHTSVNAVNLEVLPAPEYSSAQRALSDADWHFIA
DPASRFYNLVLADSGAGFFDPLTRGVLSTVSGVVVVASVSIDGAQQASVALDWLRNNGYQ
DLASRASVVINHIMPGEPNVAVKDLVRHFEQQVQPGRVVVMPWDRHIAAGTEISLDLLDPIY
KRKVLELAAALSDDFERAGRRTEQQWNFAGIEAAASAIQGNVTSIHSLLDEGKQSLTKLAAA
WGGSGSEAYQGVQQKWDATATELNNALQNLARTISEAGQAMASTEGNVTGMFATENLTV
QPERLGVLASHHDNAAVDASSGVEAAAGLGESVAITHGPYSSQFNDTTEQQWNFAGIEAA
ASAIQGNVTSIHSLLDEGKQSLTKLAAAWGGSGSEAYQGVQQKWDATATELNNALQNLAR
TISEAGQAMASTEGNVTGMFASRAFIIDPTISAIDGLYDLLGIGIPNQGGILYSSLEYFEKALE
ELAAAFPGDGWLGSAADKYAGKNRNHVNFFQELADLDRQLISLIHDQANAVQTTRDILEGA
KKGLEFVRPVAVDLTYIPVVGHALSAKTLINATQLLKLLAKLAELVAAAIADIISDVADIIKGTL
GEVWEFITNALNGLKELWDKLTGWVTGLFSRGWSNLESFFAGVPGLTGATSGLSQVTGLFG
AAGLSASSGLAHADSLASSASLPALAGIGGGSGFGGLPSLAQVHAASTRQALRPRADGPVG
AAAEQVGGQSQLVSAQGSQGMGGPVGMGGMHPSSGASKGTTTKKYSEGAAAGTEDAER
APVEADAGGGQKVLVRNVVTEQQWNFAGIEAAASAIQGNVTSIHSLLDEGKQSLTKLAAA
WGGSGSEAYQGVQQKWDATATELNNALQNLARTISEAGQAMASTEGNVTGMFAAPKTYS
EELKGTDTGQASQIQMSDPAYNINISLPSYYPDQKSLENYIAQTRDKFLSAATSSTPREAPYE
LNITSATYQSAIPPRGTQAVVLKVYQNAGGTHPTTTYKAFDWDQAYRKPITYDTLWQADTD
PLPVVFPIVQGELSKQTGQQVSIAPNAGLDPVNYQNFAVTNDGVIFFFNPGELLPEAAGPTQV
LVPRSAIDSMLAGDLVGPGSAEYAAANPTGPASVQGMSQDPVAVAASNNPELTTLTAALSG
QLNPQVNLVDTLNSGQYTVFAPTNAAFSKLPASTIDELKTNSSLLTSILTYHVVAGQTSPANV
VGTRQTLQGASVTVTGQGNSLKVGNADVVSGGVSTANATVYMIDSVLMPPAVSQDTSPKP
ATSPAAPVTTAAMADPAADLIGRGSAQYAAQNPTGPGSVAGMAQDPVATAASNNPMLSTLT
SALSGKLNPDVNLVDTLNGGEYTVFAPTNAAFDKLPAATIDQLKTDAKLLSSILTYHVIAGQA
SPSRIDGTHQTLQGADLTVIGARDDLMVNNAGLVSGGVHTANATVYMIDTVLMPPAQ
(amino acid sequence for TB antigen fusion H107e)
SEQ ID NO: 7
MQAETAVNTLFEKLEPMASILDPGASQSTTNPIFGMPSPGSSTPVGQLPPAATQTLGQLGEM
SGPMGGSGNPADEEAAQMGLLGTSPLSNHPLAGGSGPSAGAGLLRAESLPGAGGSLTRTPL
MSQLIEKPVATEQQWNFAGIEAAASAIQGNVTSIHSLLDEGKQSLTKLAAAWGGSGSEAYQ
GVQQKWDATATELNNALQNLARTISEAGQAMASTEGNVTGMFAAADYDKLFRPHEGMEAP
DDMAAQPFFDPSASFPPAPASANLPKPNGQTPPPTSDDLSERFVSAPPPPPPPPPPSPSPQRN
SGRRAERRVHPDLAAQHAAAQPDSITAATTGGRRRKRAAPDLDATQKSLRPAAKGPKVKKV
KPQKPKATKPPKVVSQRGWRHWVHALTRINLGLSPDEKYELDLHARVRRNPRGSYQIAVVG
LOGGAGKTTLTAALGSTLAQVRADRILALDADPGAGNLADRVGRQSGATIADVLAEKELSH
YNDIRAHTSVNAVNLEVLPAPEYSSAQRALSDADWHFIADPASRFYNLVLADSGAGFFDPLT
RGVLSTVSGVVVVASVSIDGAQQASVALDWLRNNGYQDLASRASVVINHIMPGEPNVAVK
DLVRHFEQQVQPGRVVVMPWDRHIAAGTEISLDLLDPIYKRKVLELAAALSDDFERAGRRTE
QQWNFAGIEAAASAIQGNVTSIHSLLDEGKQSLTKLAAAWGGSGSEAYQGVQQKWDATA
TELNNALQNLARTISEAGQAMASTEGNVTGMFATENLTVQPERLGVLASHHDNAAVDASSG
VEAAAGLGESVAITHGPYSSQFNDTTEQQWNFAGIEAAASAIQGNVTSIHSLLDEGKQSLTK
LAAAWGGSGSEAYQGVQQKWDATATELNNALQNLARTISEAGQAMASTEGNVTGMFASR
AFIIDPTISAIDGLYDLLGIGIPNQGGILYSSLEYFEKALEELAAAFPGDGWLGSAADKYAGKN
RNHVNFFQELADLDRQLISLIHDQANAVQTTRDILEGAKKGLEFVRPVAVDLTYIPVVGHALS
AKTLINATQLLKLLAKLAELVAAAIADIISDVADIIKGTLGEVWEFITNALNGLKELWDKLTGW
VTGLFSRGWSNLESFFAGVPGLTGATSGLSQVTGLFGAAGLSASSGLAHADSLASSASLPAL
AGIGGGSGFGGLPSLAQVHAASTRQALRPRADGPVGAAAEQVGGQSQLVSAQGSQGMGG
PVGMGGMHPSSGASKGTTTKKYSEGAAAGTEDAERAPVEADAGGGQKVLVRNVVTEQQW
NFAGIEAAASAIQGNVTSIHSLLDEGKQSLTKLAAAWGGSGSEAYQGVQQKWDATATELN
NALQNLARTISEAGQAMASTEGNVTGMFAAPKTYSEELKGTDTGQASQIQMSDPAYNINIS
LPSYYPDQKSLENYIAQTRDKFLSAATSSTPREAPYELNITSATYQSAIPPRGTQAVVLKVYQ
NAGGTHPTTTYKAFDWDQAYRKPITYDTLWQADTDPLPVVFPIVQGELSKQTGQQVSIAPN
AGLDPVNYQNFAVTNDGVIFFFNPGELLPEAAGPTQVLVPRSAIDSMLAGDLVGPGSAEYAA
ANPTGPASVQGMSQDPVAVAASNNPELTTLTAALSGQLNPQVNLVDTLNSGQYTVFAPTNA
AFSKLPASTIDELKTNSSLLTSILTYHVVAGQTSPANVVGTRQTLQGASVTVTGQGNSLKVG
NADVVSGGVSTANATVYMIDSVLMPPAVSQDTSPKPATSPAAPVTTAAMADPAADLIGRGS
AQYAAQNPTGPGSVAGMAQDPVATAASNNPMLSTLTSALSGKLNPDVNLVDTLNGGEYTVF
APTNAAFDKLPAATIDQLKTDAKLLSSILTYHVIAGQASPSRIDGTHQTLQGADLTVIGARDD
LMVNNAGLVSGGVHTANATVYMIDTVLMPPAQ
(amino acid sequence for malaria antigen fusion of Pro + I-6C)
SEQ ID NO: 8
EYVDEKERQGEIYPFGDEEEKDEGGESFTYEKSEVDKTDLFKFIEGGEGDDVYKVDGSKVLL
DDDTISRVSKKHTARDGEYGEYGEAVEDGENVIKIIRSVLQSGALPSVGVDELDKIDLSYET
TESGDTAVSEDSYDKYASNNTNKERSYVCDFTDQLKPTESGPKVKKCEVKVNEPLIKVKIIC
PLKGSVEKLYDNIEYVPKKSPYVVLTKEETKLKEKLLSKLIYGLLISPTVNEKENNFKEGVIEFT
LPPVVHKATVFYFICDNSKTEDDNKKGNRGIVEVYVEPYGNKINGRSEKKVIHGCNFSSNVS
SKHTFTDSLDISLVDDSAHISCNVHLSEPKYNHLVGLNCPGDIIPDCFFQVYQPESEELEPSN
IVYLDSQINIGDIEYYEDAEGDDKIKLFGIVGSIPKTTSFTCICKKDKKSAYMTVTIDSA
(amino acid sequence for Influenza antigen fusion of HA,
NA, NP, M1 and M2)
SEQ ID NO: 9
MKAILVVLLYTFATANADTLCIGYHANNSTDTVDTVLEKNVTVTHSVNLLEDKHNGKLCKLR
GVAPLHLGKCNIAGWILGNPECESLSTASSWSYIVETPSSDNGTCYPGDFIDYEELREQLSS
VSSFERFEIFPKTSSWPNHDSDKGVTAACPHAGAKSFYKNLIWLVKKGNSYPKLSKSYINDK
GKEVLVLWGIHHPSTSADQQSLYQNADTYVFVGSSRYSKKFKPEIAIRPKVRDQEGRMNYY
WTLVEPGDKITFEATGNLVVPRYAFAMERNAGSGIIISDTPVHDCNTTCQTPKGAINTSLPFQ
NIHPITIGKCPKYVKSTKLRLATGLRNIPSIQSRGLFGAIAGFIEGGWTGMVDGWYGYHHQN
EQGSGYAADLKSTQNAIDEITNKVNSVIEKMNTQFTAVGKEFNHLEKRIENLNKKVDDGFLD
IWTYNAELLVLLENERTLDYHDSNVKNLYEKVRSQLKNNAKEIGNGCFEFYHKCDNTCMESV
KNGTYDYPKYSEEAKLNREEIDGVKLESTRIYQILAIYSTVASSLVLVVSLGAISFWMCSNGS
LQCRICIRRKRGSGATNFSLLKQAGDVEENPGPMKTIIALSYIFCLALGQDLPGNDNSTATLC
LGHHAVPNGTLVKTITDDQIEVTNATELVQSSSTGKICNNPHRILDGIDCTLIDALLGDPHCD
VFQNETWDLFVERSKAFSNCYPYDVPDYASLRSLVASSGTLEFITEGFTWTGVTQNGGSNA
CKRGPGSGFFSRLNWLTKSGSTYPVLNVTMPNNDNFDKLYIWGVHHPSTNQEQTSLYVQA
SGRVTVSTRRSQQTIIPNIESRPWVRGLSSRISIYWTIVKPGDVLVINSNGNLIAPRGYFKMR
TGKSSIMRSDAPIDTCISECITPNGSIPNDKPFQNVNKITYGACPKYVKQNTLKLATGMRNVP
EKQTRGLFGAIAGFIENGWEGMIDGWYGFRHQNSEGTGQAADLKSTQAAIDQINGKLNRV
IEKTNEKFHQIEKEFSEVEGRIQDLEKYVEDTKIDLWSYNAELLVALENQHTIDLTDSEMNKL
FEKTRRQLRENAEDMGNGCFKIYHKCDNACIESIRNGTYDHDVYRDEALNNRFQIKGVELK
SGYKDWILWISFAISCFLLCVVLLGFIMWACQRGNIRCNICIRRKRGSGATNFSLLKQAGDV
EENPGPMNPNQKIITIGSVCMTIGMANLILQIGNIISIWISHSIQLGNQNQIETCNQSVITYEN
NTWVNQTYVNISNTNFAAGQSVVSVKLAGNSSLCPVSGWAIYSKDNSVRIGSKGDVFVIRE
PFISCSPLECRTFFLTQGALLNDKHSNGTIKDRSPYRTLMSCPIGEVPSPYNSRFESVAWSAS
ACHDGINWLTIGISGPDNGAVAVLKYNGIITDTIKSWRNNILRTQESECACVNGSCFTVMTD
GPSNGQASYKIFRIEKGKIVKSVEMNAPNYHYEECSCYPDSSEITCVCRDNWHGSNRPWVS
FNQNLEYQIGYICSGIFGDNPRPNDKTGSCGPVSSNGANGVKGFSFKYGNGVWIGRTKSIS
SRNGFEMIWDPNGWTGTDNNFSIKQDIVGINEWSGYSGSFVQHPELTGLDCIRPCFWVELI
RGRPKENTIWTSGSSISFCGVNSDTVGWSWPDGAELPFTIDKRRKRGSGATNFSLLKQAG
DVEENPGPMNPNQKIITIGSVSLTIATVCFLMQIAILVTTVTLHFKQYECDSPASNQVMPCEPI
IIERNITEIVYLNNTTIEKEICPKVVEYRNWSKPQCQITGFAPFSKDNSIRLSAGGDIWVTREP
YVSCDHGKCYQFALGQGTTLDNKHSNDTIHDRIPHRTLLMNELGVPFHLGTRQVCIAWSSS
SCHDGKAWLHVCITGDDKNATASFIYDGRLVDSIGSWSQNILRTQESECVCINGTCTVVMT
DGSASGRADTRILFIEEGKIVHISPLSGSAQHVEECSCYPRYPGVRCICRDNWKGSNRPVVD
INMEDYSIDSSYVCSGLVGDTPRNDDRSSNSNCRNPNNERGNQGVKGWAFDNGDDVWM
GRTISKDLRSGYETFKVIGGWSTPNSKSQINRQVIVDSDNRSGYSGIFSVEGKSCINRCFYV
ELIRGRKQETRVWWTSNSIVVFCGTSGTYGTGSWPDGANINFMPIRRKRGSGATNFSLLKQ
AGDVEENPGPMASQGTKRSYEQMETDGERQNATEIRASVGRMIGGIGRFYIQMCTELKLSD
YEGRLIQNSITIERMVLSAFDERRNKYLEEHPSAGKDPKKTGGPIYRRIDGKWMRELILWEK
DDIKRIYKQANNGEDATAGLTHMMIWHSNLNDATYQRTRALVRTGMDPRMCSLMQGSTLP
RRSGAAGAAVKGVGTMVMELIRMIKRGINDRNFWRGENGRRTRIAYERMCNILKGKFQTA
AQRAMMDQVRESRNPGNAEIEDLIFLARSALILRGSVAHKSCLPACVYGPAVASGYDFEREG
YSLVGIDPFRLLQNSQVYSLIRPNENPAHKSQLVWMACHSAAFEDLRVSSFIRGTRVVPRGK
LSTRGVQIASNENMETMDSSTLELRSRYWAIRTRSGGNTNQQRASAGQISVQPTFSVQRNL
PFERATIMAAFTGNTEGRTRDMRTEIIRMMESARPEDVSFQGRGVFELSDEKATSPIVPSFD
MSNEGSYFFGDNAEEYDNRRKRGSGATNFSLLKQAGDVEENPGPMSLLTEVETYVLSIVPS
GPLKAEIAQRLEDVFAGKNTDLEALMEWLKTRPILSPLTKGILGFVFTLTVPSERGLQRRRFV
QNALNGNGDPNNMDRAVKLYRKLKREITFHGAKEVALSYSAGALASCMGLIYNRMGTVTTE
VAFGLVCATCEQIADSQHRSHRQMVTTTNPLIRHENRMVLASTTAKAMEQMAGSSEQAAE
AMEVASQARQMVQAMRTIGTHPSSSAGLKDDLIENLQAYQKRMGVQMQRFKRRKRGSGA
TNFSLLKQAGDVEENPGPMSLLTEVETPTRNEWGCRCNDSSDPLVIAASIIGILHLILWILDR
LFFKCIYRRLKYGLKRGPSTEGVPESMREEYRKEQQSAVDVDDGHFVNIELE
(amino acid sequence for SARS-CoV-2 SPIKE protein)
SEQ ID NO: 10
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSYLTP
GDSSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTGVLTESNKKFLPF
QQFGRDIADTTDAVRDPQTLEILDITPCSFGGVSVITPGTNTSNQVAVLYQDVNCTEVPVAI
HADQLTPTWRVYSTGSNVFQTRAGCLIGAEHVNNSYECDIPIGAGICASYQTQTNSPRRAR
SVASQSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLPPLLTDEMIAQYTSA
LLAGTITSGWTFGAGAALQIPFAMQMAYRFNGIGVTQNVLYENQKLIANQFNSAIGKIQDSL
SSTASALGKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRL
NEVAKNLNESLIDLQELGKYEQYIKWPWYIWLGFIAGLIAIVMVTIMLCCMTSCCSCLKGCC
SCGSCCKFDEDDSEPVLKGVKLHYT
(amino acid sequence for SARS-COV-2 S1 protein)
SEQ ID NO: 11
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATN
VVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSYLTP
GDSSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTGVLTESNKKFLPF
QQFGRDIADTTDAVRDPQTLEILDITPCSFGGVSVITPGTNTSNQVAVLYQDVNCTEVPVAI
HADQLTPTWRVYSTGSNVFQTRAGCLIGAEHVNNSYECDIPIGAGICASYQTQTNSPRRAR
(amino acid sequence for SARS-COV-2 S2 protein)
SEQ ID NO: 12
SVASQSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLPPLLTDEMIAQYTSA
LLAGTITSGWTFGAGAALQIPFAMQMAYRFNGIGVTQNVLYENQKLIANQFNSAIGKIQDSL
SSTASALGKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQSAPHGVV
FLHVTYVPAQEKNFTTAPAICHDGKAHFPREGVFVSNGTHWFVTQRNFYEPQIITTDNTFVS
GNCDVVIGIVNNTVYDPLQPELDSFKEELDKYFKNHTSPDVDLGDISGINASVVNIQKEIDRL
NEVAKNLNESLIDLQELGKYEQYIKWPWYIWLGFIAGLIAIVMVTIMLCCMTSCCSCLKGCC
SCGSCCKFDEDDSEPVLKGVKLHYT
(Amino acid sequence for CTH522 recombinant Chlamydia antigen)
SEQ ID NO: 13
MKDAISMRVGYYGDFVFDRVLKTDVNKEFQMGAKPTTDTGNSAAPSTLTARENPAYGRHM
QDAEMFTNAASMALNIWDRFDVFSTLGATSGYLKGNSASFNLVGLFGDNENQKTVKAESVP
NMSFDQSVVELYTDTTFAWSVGARAALWESGSATLGASFQYAQSKPKVEELNVLSNAAEFT
INKPKGYVGKEFPLDLTAGTDAATGTKDASIDYHEWQASLALSYRLNMFTPYIGVKWSRASF
DADTIRIAQPKSATAIFDTTTLNPTIAGAGDVKTGAEGQLGDTMQIVSLQLNNMFTPYIGVK
WSRASFDADTIRIAQPKSATAIFDTTTLNPTIAGAGDVKASAEGQLGDTMQIVSLQLNNMFT
PYIGVKWSRASFDSDTIRIAQPRLVTPVVDITTLNPTIAGSGSVAGANTEGQISDTMQIVSLQ
LNNMFTPYIGVKWSRASFDSNTIRIAQPKLAKPVVDITTLNPTIAGSGSVVAANSEGQISDT
MQIVSLQLN
(Influenza A H1N1 (A/Puerto Rico/8/1934) Hemagglutinin
Protein sequence)
SEQ ID NO: 14
MKANLLVLLCALAAADADTICIGYHANNSTDTVDTVLEKNVTVTHSVNLLEDSHNGKLCR
LKGIAPLQLGKCNIAGWLLGNPECDPLLPVRSWSYIVETPNSENGICYPGDFIDYEELRE
QLSSVSSFERFEIFPKESSWPNHNTNGVTAACSHEGKSSFYRNLLWLTEKEGSYPKLKNS
YVNKKGKEVLVLWGIHHPPNSKEQQNLYQNENAYVSVVTSNYNRRFTPEIAERPKVRDQA
GRMNYYWTLLKPGDTIIFEANGNLIAPMYAFALSRGFGSGIITSNASMHECNTKCQTPLG
AINSSLPYQNIHPVTIGECPKYVRSAKLRMVTGLRNIPSIQSR

1. An adjuvant composition comprising dimethyldioctadecyl ammonium salt (DDA), monomycoloyl glycerol (MMG), and the CpG ODN 2006 oligodeoxynucleotide having SEQ ID NO:1 or a sequence having 90% identity to SEQ ID NO:1.

2. The adjuvant composition according to claim 1, said composition comprising 1000-4000 μg/ml DDA,
or
2000-3000 μg/ml DDA,
or 2500 μg/ml DDA
and, or
wherein the dimethyldioctadecyl ammonium salt is dimethyldioctadecyl ammonium bromide.

3. The adjuvant composition according to claim 1, wherein
the composition comprises 100-1000 μg/ml MMG,
or
300-700 μg/ml MMG,
or
500 μg/ml MMG.

4. The adjuvant composition according to claim 1, wherein the composition comprises 2500 μg/ml DDA and 500 μg/ml MMG.

5. The adjuvant composition according to claim 1, wherein the composition comprises the CpG ODN 2006 oligodeoxynucleotide having SEQ ID NO: 1 or a sequence having 90% identity to SEQ ID NO:1 in an amount selected from 10-500 μg/ml, 20-300 μg/ml, or 50-200 μg/ml.

6. The adjuvant composition according to claim 1, wherein the composition comprises 2500 μg/ml DDA and 500 μg/ml MMG, and 100 μg/ml CpG ODN 2006 oligodeoxynucleotide having SEQ ID NO:1 or a sequence having 90% identity to SEQ ID NO:1.

7. The adjuvant composition according to claim 1, wherein the CpG ODN 2006 oligodeoxynucleotide has a phosphorothioate backbone.

8. A vaccine comprising the adjuvant composition according to claim 1 and at least one antigen wherein the vaccine induces one or more of a cell mediated immune response and production antigen-specific antibodies.

9. (canceled)

10. The vaccine according to claim 8, wherein the antigen is an antigen from a pathogen causing an infectious disease.

11. The vaccine according to claim 10, wherein the antigen is selected from the group comprising influenza antigens, coronavirus antigens, tuberculosis antigens, malaria antigens, and chlamydia antigens.

12. The vaccine according to claim 11, wherein
i) the tuberculosis antigen is a fusion protein selected from H56 having SEQ ID NO: 3, H107 having SEQ ID NO: 4, H107b having SEQ ID NO: 5, H107c having SEQ ID NO: 6 and H107e having SEQ ID NO: 7 or a fusion protein having 90% sequence identity to any one of SEQ ID NOS: 3, 4, 5, 6 or 7; or
ii) the malaria antigen is a fusion protein comprising an antigenic domain Pro and I of fragment of Pfs230 and the antigenic domain 6C of Pfs48/45 having SEQ ID NO: 8 or a fusion protein having 90% sequence identity to SEQ ID NO: 8; or
iii) the influenza antigen is a protein selected from the group comprising antigens of influenza A or B viruses, or a fusion protein of the influenza antigens HA, NA, NP, M1 and M2 having SEQ ID NO:9 or a fusion protein having 90% sequence identity to SEQ ID NO: 9; or
iv) the coronavirus antigen is a protein selected from the group comprising antigens of alpha, beta, gamma, or delta coronaviruses, selected from Spike from SARS-COV-2 having SEQ ID NO:10, Spike S1 from SARS-COV-2 having SEQ ID NO: 11, or Spike S2 from SARS-COV-2 having SEQ ID NO: 12 or a protein having 90% sequence identity to any one of SEQ ID NOS: 10, 11 or 12; or
v) the chlamydia antigen is the CTH522 antigen having SEQ ID NO: 13.

13. (canceled)

14. (canceled)

15. (canceled)

16. (canceled)

17. A method for the treatment or inhibition of an infectious disease comprising administration of an effective amount of a vaccine according to claim 12.

18. The method of claim 17, wherein said infections disease is tuberculosis and said vaccine is a fusion protein of tuberculosis antigens.

19. The method of claim 17, wherein said infectious disease is malaria and said vaccine is a fusion protein of malaria antigens.

20. The method of claim 17, wherein said infectious disease is influenza and said vaccine is a fusion protein of influenza antigens.

21. The method of claim 17, wherein said infectious disease is SARS-COV-2 infection and said vaccine comprises an antigen selected from spike protein, a spike S1 protein and a spike S2 protein.

22. A method for treatment of an infectious disease comprising administration of the vaccine of claim 17, wherein the vaccine is administered to a subject via a route selected from intradermal, intravenous, intramuscular or subcutaneous injection.

23. The method according to claim 22, wherein the subject is selected from humans, primates, mammals, cynomolgus monkeys, rhesus monkeys cattle, pigs, horses, sheep, goats, mink, ferrets, hamsters, cats and dogs, and birds.

24. (canceled)

25. The method according to claim 12, wherein the vaccine is administered as one dose, or as two doses, or as three doses.

26. (canceled)


SEQUENCE LISTING (14 sequences)

SEQ ID NO: 1 - DNA, 24 bp, synthetic construct
  source 1..24: mol_type = other DNA
tcgtcgttttgtcgttttgtcgtt

SEQ ID NO: 2 - DNA, 20 bp, synthetic construct
  source 1..20: mol_type = other DNA
tccatgacgttcctgacgtt

SEQ ID NO: 3 - protein, 454 aa, synthetic construct
MSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGTEQQWNFAGIEAAASAIQGNVTSIHSLLDEGKQSLTKLAAAWGGSGSEAYQGVQQKWDATATELNNALQNLARTISEAGQAMASTEGNVTGMFAVIAGVDQALAATGQASQRAAGASGGVTVGVGVGTEQRNLSVVAPSQFTFSSRSPDFVDETAGQSWCAILGLNQFH

SEQ ID NO: 4 - protein, 2153 aa, synthetic construct
MQAETAVNTLFEKLEPMASILDPGASQSTTNPIFGMPSPGSSTPVGQLPPAATQTLGQLGEMSGPMGGSGNPADEEAAQMGLLGTSPLSNHPLAGGSGPSAGAGLLRAESLPGAGGSLTRTPLMSQLIEKPVATEQQWNFAGIEAAASAIQGNVTSIHSLLDEGKQSLTKLAAAWGGSGSEAYQGVQQKWDATATELNNALQNLARTISEAGQAMASTEGNVTGMFAAADYDKLFRPHEGMEAPDDMAAQPFFDPSASFPPAPASANLPKPNGQTPPPTSDDLSERFVSAPPPPPPPPPPPPPTPMPIAAGEPPSPEPAASKPPTPPMPIAGPEPAPPKPPTPPMPIAGPEPAPPKPPTPPMPIAGPAPTPTESQLAPPRPPTPQTPTGAPQQPESPAPHVPSHGPHQPRRTAPAPPWAKMPIGEPPPAPSRPSASPAEPPTRPAPQHSRRARRGHRYRTDTERNVGKVATGPSIQARLRAEEASGAQLAPGTEPSPAPLGQPRSYLAPPTRPAPTEPPPSPSPQRNSGRRAERRVHPDLAAQHAAAQPDSITAATTGGRRRKRAAPDLDATQKSLRPAAKGPKVKKVKPQKPKATKPPKVVSQRGWRHWVHALTRINLGLSPDEKYELDLHARVRRNPRGSYQIAVVGLQGGAGKTTLTAALGSTLAQVRADRILALDADPGAGNLADRVGRQSGATIADVLAEKELSHYNDIRAHTSVNAVNLEVLPAPEYSSAQRALSDADWHFIADPASRFYNLVLADSGAGFFDPLTRGVLSTVSGVVVVASVSIDGAQQASVALDWLRNNGYQDLASRASVVINHIMPGEPNVAVKDLVRHFEQQVQPGRVVVMPWDRHIAAGTEISLDLLDPIYKRKVLELAAALSDDFERAGRRTEQQWNFAGIEAAASAIQGNVTSIHSLLDEGKQSLTKLAAAWGGSGSEAYQGVQQKWDATATELNNALQNLARTISEAGQAMASTEGNVTGMFATENLTVQPERLGVLASHHDNAAVDASSGVEAAAGLGESVAITHGPYSSQFNDTTEQQWNFAGIEAAASAIQGNVTSIHSLLDEGKQSLTKLAAAWGGSGSEAYQGVQQKWDATATELNNALQNLARTISEAGQAMASTEGNVTGMFASRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVTEQQWNFAGIEAAASAIQGNVTSIHSLLDEGKQSLTKLAAAWGGSGSEAYQGVQQKWDATATELNNALQNLARTISEAGQAMASTEGNVTGMFAAPKTYSEELKGTDTGQASQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAGDLVGPGSAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVSGGVSTANATVYMIDSVLMPPAVSQDTSPKPATSPAAPVTTAAMADPAADLIGRGSAQYAAQNPTGPGSVAGMAQDPVATAASNNPMLSTLTSALSGKLNPDVNLVDTLNGGEYTVFAPTNAAFDKLPAATIDQLKTDAKLLSSILTYHVIAGQASPSRIDGTHQTLQGADLTVIGARDDLMVNNAGLVSGGVHTANATVYMIDTVLMPPAQ

SEQ ID NO: 5 - protein, 2100 aa, synthetic construct
MTEQQWNFAGIEAAASAIQGNVTSIHSLLDEGKQSLTKLAAAWGGSGSEAYQGVQQKWDATATELNNALQNLARTISEAGQAMASTEGNVTGMFAQAETAVNTLFEKLEPMASILDPGASQSTTNPIFGMPSPGSSTPVGQLPPAATQTLGQLGEMSGPMGGSGNPADEEAAQMGLLGTSPLSNHPLAGGSGPSAGAGLLRAESLPGAGGSLTRTPLMSQLIEKPVATEQQWNFAGIEAAASAIQGNVTSIHSLLDEGKQSLTKLAAAWGGSGSEAYQGVQQKWDATATELNNALQNLARTISEAGQAMASTEGNVTGMFAAADYDKLFRPHEGMEAPDDMAAQPFFDPSASFPPAPASANLPKPNGQTPPPTSDDLSERFVSAPPPPPPPPPPPPPTPMPIAAGEPPSPEPAASKPPTPPMPIAGPEPAPPKPPTPPMPIAGPEPAPPKPPTPPMPIAGPAPTPTESQLAPPRPPTPQTPTGAPQQPESPAPHVPSHGPHQPRRTAPAPPWAKMPIGEPPPAPSRPSASPAEPPTRPAPQHSRRARRGHRYRTDTERNVGKVATGPSIQARLRAEEASGAQLAPGTEPSPAPLGQPRSYLAPPTRPAPTEPPPSPSPQRNSGRRAERRVHPDLAAQHAAAQPDSITAATTGGRRRKRAAPDLDATQKSLRPAAKGPKVKKVKPQKPKATKPPKVVSQRGWRHWVHALTRINLGLSPDEKYELDLHARVRRNPRGSYQIAVVGLQGGAGKTTLTAALGSTLAQVRADRILALDADPGAGNLADRVGRQSGATIADVLAEKELSHYNDIRAHTSVNAVNLEVLPAPEYSSAQRALSDADWHFIADPASRFYNLVLADSGAGFFDPLTRGVLSTVSGVVVVASVSIDGAQQASVALDWLRNNGYQDLASRASVVINHIMPGEPNVAVKDLVRHFEQQVQPGRVVVMPWDRHIAAGTEISLDLLDPIYKRKVLELAAALSDDFERAGRRTEQQWNFAGIEAAASAIQGNVTSIHSLLDEGKQSLTKLAAAWGGSGSEAYQGVQQKWDATATELNNALQNLARTISEAGQAMASTEGNVTGMFASRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVTEQQWNFAGIEAAASAIQGNVTSIHSLLDEGKQSLTKLAAAWGGSGSEAYQGVQQKWDATATELNNALQNLARTISEAGQAMASTEGNVTGMFAAPKTYSEELKGTDTGQASQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAGDLVGPGSAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVSGGVSTANATVYMIDSVLMPPAVSQDTSPKPATSPAAPVTTAAMADPAADLIGRGSAQYAAQNPTGPGSVAGMAQDPVATAASNNPMLSTLTSALSGKLNPDVNLVDTLNGGEYTVFAPTNAAFDKLPAATIDQLKTDAKLLSSILTYHVIAGQASPSRIDGTHQTLQGADLTVIGARDDLMVNNAGLVSGGVHTANATVYMIDTVLMPPAQ

SEQ ID NO: 6 - protein, 1771 aa, synthetic construct
MQAETAVNTLFEKLEPMASILDPGASQSTTNPIFGMPSPGSSTPVGQLPPAATQTLGQLGEMSGPMGGSGNPADEEAAQMGLLGTSPLSNHPLAGGSGPSAGAGLLRAESLPGAGGSLTRTPLMSQLIEKPVATEQQWNFAGIEAAASAIQGNVTSIHSLLDEGKQSLTKLAAAWGGSGSEAYQGVQQKWDATATELNNALQNLARTISEAGQAMASTEGNVTGMFAWVHALTRINLGLSPDEKYELDLHARVRRNPRGSYQIAVVGLQGGAGKTTLTAALGSTLAQVRADRILALDADPGAGNLADRVGRQSGATIADVLAEKELSHYNDIRAHTSVNAVNLEVLPAPEYSSAQRALSDADWHFIADPASRFYNLVLADSGAGFFDPLTRGVLSTVSGVVVVASVSIDGAQQASVALDWLRNNGYQDLASRASVVINHIMPGEPNVAVKDLVRHFEQQVQPGRVVVMPWDRHIAAGTEISLDLLDPIYKRKVLELAAALSDDFERAGRRTEQQWNFAGIEAAASAIQGNVTSIHSLLDEGKQSLTKLAAAWGGSGSEAYQGVQQKWDATATELNNALQNLARTISEAGQAMASTEGNVTGMFATENLTVQPERLGVLASHHDNAAVDASSGVEAAAGLGESVAITHGPYSSQFNDTTEQQWNFAGIEAAASAIQGNVTSIHSLLDEGKQSLTKLAAAWGGSGSEAYQGVQQKWDATATELNNALQNLARTISEAGQAMASTEGNVTGMFASRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVTEQQWNFAGIEAAASAIQGNVTSIHSLLDEGKQSLTKLAAAWGGSGSEAYQGVQQKWDATATELNNALQNLARTISEAGQAMASTEGNVTGMFAAPKTYSEELKGTDTGQASQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAGDLVGPGSAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVSGGVSTANATVYMIDSVLMPPAVSQDTSPKPATSPAAPVTTAAMADPAADLIGRGSAQYAAQNPTGPGSVAGMAQDPVATAASNNPMLSTLTSALSGKLNPDVNLVDTLNGGEYTVFAPTNAAFDKLPAATIDQLKTDAKLLSSILTYHVIAGQASPSRIDGTHQTLQGADLTVIGARDDLMVNNAGLVSGGVHTANATVYMIDTVLMPPAQ

SEQ ID NO: 7 - protein, 1933 aa, synthetic construct
MQAETAVNTLFEKLEPMASILDPGASQSTTNPIFGMPSPGSSTPVGQLPPAATQTLGQLGEMSGPMGGSGNPADEEAAQMGLLGTSPLSNHPLAGGSGPSAGAGLLRAESLPGAGGSLTRTPLMSQLIEKPVATEQQWNFAGIEAAASAIQGNVTSIHSLLDEGKQSLTKLAAAWGGSGSEAYQGVQQKWDATATELNNALQNLARTISEAGQAMASTEGNVTGMFAAADYDKLFRPHEGMEAPDDMAAQPFFDPSASFPPAPASANLPKPNGQTPPPTSDDLSERFVSAPPPPPPPPPPSPSPQRNSGRRAERRVHPDLAAQHAAAQPDSITAATTGGRRRKRAAPDLDATQKSLRPAAKGPKVKKVKPQKPKATKPPKVVSQRGWRHWVHALTRINLGLSPDEKYELDLHARVRRNPRGSYQIAVVGLQGGAGKTTLTAALGSTLAQVRADRILALDADPGAGNLADRVGRQSGATIADVLAEKELSHYNDIRAHTSVNAVNLEVLPAPEYSSAQRALSDADWHFIADPASRFYNLVLADSGAGFFDPLTRGVLSTVSGVVVVASVSIDGAQQASVALDWLRNNGYQDLASRASVVINHIMPGEPNVAVKDLVRHFEQQVQPGRVVVMPWDRHIAAGTEISLDLLDPIYKRKVLELAAALSDDFERAGRRTEQQWNFAGIEAAASAIQGNVTSIHSLLDEGKQSLTKLAAAWGGSGSEAYQGVQQKWDATATELNNALQNLARTISEAGQAMASTEGNVTGMFATENLTVQPERLGVLASHHDNAAVDASSGVEAAAGLGESVAITHGPYSSQFNDTTEQQWNFAGIEAAASAIQGNVTSIHSLLDEGKQSLTKLAAAWGGSGSEAYQGVQQKWDATATELNNALQNLARTISEAGQAMASTEGNVTGMFASRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVTEQQWNFAGIEAAASAIQGNVTSIHSLLDEGKQSLTKLAAAWGGSGSEAYQGVQQKWDATATELNNALQNLARTISEAGQAMASTEGNVTGMFAAPKTYSEELKGTDTGQASQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAGDLVGPGSAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVSGGVSTANATVYMIDSVLMPPAVSQDTSPKPATSPAAPVTTAAMADPAADLIGRGSAQYAAQNPTGPGSVAGMAQDPVATAASNNPMLSTLTSALSGKLNPDVNLVDTLNGGEYTVFAPTNAAFDKLPAATIDQLKTDAKLLSSILTYHVIAGQASPSRIDGTHQTLQGADLTVIGARDDLMVNNAGLVSGGVHTANATVYMIDTVLMPPAQ

SEQ ID NO: 8 - protein, 436 aa, synthetic construct
EYVDEKERQGEIYPFGDEEEKDEGGESFTYEKSEVDKTDLFKFIEGGEGDDVYKVDGSKVLLDDDTISRVSKKHTARDGEYGEYGEAVEDGENVIKIIRSVLQSGALPSVGVDELDKIDLSYETTESGDTAVSEDSYDKYASNNTNKERSYVCDFTDQLKPTESGPKVKKCEVKVNEPLIKVKIICPLKGSVEKLYDNIEYVPKKSPYVVLTKEETKLKEKLLSKLIYGLLISPTVNEKENNFKEGVIEFTLPPVVHKATVFYFICDNSKTEDDNKKGNRGIVEVYVEPYGNKINGRSEKKVIHGCNFSSNVSSKHTFTDSLDISLVDDSAHISCNVHLSEPKYNHLVGLNCPGDIIPDCFFQVYQPESEELEPSNIVYLDSQINIGDIEYYEDAEGDDKIKLFGIVGSIPKTTSFTCICKKDKKSAYMTVTIDSA

SEQ ID NO: 9 - protein, 3073 aa, synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSDKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICIRRKRGSGATNFSLLKQAGDVEENPGPMKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIESRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICIRRKRGSGATNFSLLKQAGDVEENPGPMNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDKRRKRGSGATNFSLLKQAGDVEENPGPMNPNQKIITIGSVSLTIATVCFLMQIAILVTTVTLHFKQYECDSPASNQVMPCEPIIIERNITEIVYLNNTTIEKEICPKVVEYRNWSKPQCQITGFAPFSKDNSIRLSAGGDIWVTREPYVSCDHGKCYQFALGQGTTLDNKHSNDTIHDRIPHRTLLMNELGVPFHLGTRQVCIAWSSSSCHDGKAWLHVCITGDDKNATASFIYDGRLVDSIGSWSQNILRTQESECVCINGTCTVVMTDGSASGRADTRILFIEEGKIVHISPLSGSAQHVEECSCYPRYPGVRCICRDNWKGSNRPVVDINMEDYSIDSSYVCSGLVGDTPRNDDRSSNSNCRNPNNERGNQGVKGWAFDNGDDVWMGRTISKDLRSGYETFKVIGGWSTPNSKSQINRQVIVDSDNRSGYSGIFSVEGKSCINRCFYVELIRGRKQETRVWWTSNSIVVFCGTSGTYGTGSWPDGANINFMPIRRKRGSGATNFSLLKQAGDVEENPGPMASQGTKRSYEQMETDGERQNATEIRASVGRMIGGIGRFYIQMCTELKLSDYEGRLIQNSITIERMVLSAFDERRNKYLEEHPSAGKDPKKTGGPIYRRIDGKWMRELILWEKDDIKRIYKQANNGEDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGPAVASGYDFEREGYSLVGIDPFRLLQNSQVYSLIRPNENPAHKSQLVWMACHSAAFEDLRVSSFIRGTRVVPRGKLSTRGVQIASNENMETMDSSTLELRSRYWAIRTRSGGNTNQQRASAGQISVQPTFSVQRNLPFERATIMAAFTGNTEGRTRDMRTEIIRMMESARPEDVSFQGRGVFELSDEKATSPIVPSFDMSNEGSYFFGDNAEEYDNRRKRGSGATNFSLLKQAGDVEENPGPMSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSAGALASCMGLIYNRMGTVTTEVAFGLVCATCEQIADSQHRSHRQMVTTTNPLIRHENRMVLASTTAKAMEQMAGSSEQAAEAMEVASQARQMVQAMRTIGTHPSSSAGLKDDLIENLQAYQKRMGVQMQRFKRRKRGSGATNFSLLKQAGDVEENPGPMSLLTEVETPTRNEWGCRCNDSSDPLVIAASIIGILHLILWILDRLFFKCIYRRLKYGLKRGPSTEGVPESMREEYRKEQQSAVDVDDGHFVNIELE

SEQ ID NO: 10 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 685 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 12 - protein, 588 aa, synthetic construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 502 aa, synthetic construct
MKDAISMRVGYYGDFVFDRVLKTDVNKEFQMGAKPTTDTGNSAAPSTLTARENPAYGRHMQDAEMFTNAASMALNIWDRFDVFSTLGATSGYLKGNSASFNLVGLFGDNENQKTVKAESVPNMSFDQSVVELYTDTTFAWSVGARAALWESGSATLGASFQYAQSKPKVEELNVLSNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNNMFTPYIGVKWSRASFDADTIRIAQPKSATAIFDTTTLNPTIAGAGDVKASAEGQLGDTMQIVSLQLNNMFTPYIGVKWSRASFDSDTIRIAQPRLVTPVVDITTLNPTIAGSGSVAGANTEGQISDTMQIVSLQLNNMFTPYIGVKWSRASFDSNTIRIAQPKLAKPVVDITTLNPTIAGSGSVVAANSEGQISDTMQIVSLQLN

SEQ ID NO: 14 - protein, 343 aa, synthetic construct
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSR